(19) United States
(12) Patent Application Publication
(10) Pub. No.: US 2020/0071767 A1
(43) Pub. Date: Mar. 5, 2020

(54) BARRETT'S ESOPHAGUS PROGRESSION TO CANCER GENE PANEL AND METHODS OF USE THEREOF

(71) Applicant: THE TRUSTEES OF COLUMBIA UNIVERSITY IN THE CITY OF NEW YORK, New York, NY (US)

(72) Inventors: Antonia R. Sepulveda, New York, NY (US); Jorge L. Sepulveda, New York, NY (US)

(21) Appl. No.: 16/379,531

(22) Filed: Apr. 9, 2019

Related U.S. Application Data
(60) Provisional application No. 62/654,918, filed on Apr. 9, 2018.

(51) Int. Cl. C12Q 1/6886; C12Q 1/6827; G16H 50/30

(52) U.S. Cl. CPC C12Q 1/6886; C12Q 1/6827; G16H 50/30; C12Q 2539/10; C12Q 2600/154; C12Q 2600/118; C12Q 2600/112; C12Q 2600/156

(57) ABSTRACT

The present invention provides, inter alia, methods of predicting the risk of a subject having Barrett's Esophagus to develop a more severe condition, such as, e.g., low-grade dysplasia (LGD), high-grade dysplasia (HGD) and esophageal adenocarcinoma (EAC), methods for supporting the diagnosis of dysplasia or esophageal adenocarcinoma (EAC) in a subject having Barrett's Esophagus. Also provided are kits to implement such methods.

### CROSS REFERENCE TO RELATED APPLICATIONS

The present application claims benefit of U.S. Provisional Patent Application Ser. No. 62/654,918, filed on Apr. 9, 2018 which application is incorporated by reference herein in its entirety.

### GOVERNMENT FUNDING

This invention was made with government support under CA208711 and CA163004 awarded by the National Institutes of Health. The government has certain rights in the invention.

### FIELD OF INVENTION

The present invention provides, inter alia, methods of predicting the risk of a subject having Barrett's Esophagus to develop a more severe condition, such as, e.g., low-grade dysplasia (LGD), high-grade dysplasia (HGD) and esophageal adenocarcinoma (EAC), methods for supporting the diagnosis of dysplasia or esophageal adenocarcinoma (EAC) in a subject having Barrett's Esophagus. Also provided are kits to implement such methods.

### BACKGROUND OF THE INVENTION

Esophageal adenocarcinoma (EAC) has increased greater than 10-fold over the past decades in the U.S. (Pohl et al 2010), increasing as a cause of cancer morbidity and mortality (Rustgi et al 2014; Snider et al 2016; Pohl et al 2010). EAC represents the 7th leading cause of cancer deaths in men (Siegel et al 2018). Unfortunately, the overall 5-year survival of patients with EAC is below 19% (Cancer Stat Facts: Esophageal Cancer. https://seer.cancer.gov/statfacts.html/esoph.html), with most tumors discovered in advanced stages, after local invasion and/or metastasis occurred (Rustgi et al 2014; Snider et al 2016). Most EACs arise in patients with Barrett's esophagus (BE) (Ruol et al 2000), which is characterized by replacement of the normal squamous esophageal mucosa by columnar epithelium with intestinal metaplasia (Barrett's intestinal metaplasia—BIM) and may subsequently progress to dysplasia (low-grade dysplasia—LGD or high-grade dysplasia—HGD), intramucosal adenocarcinoma and advanced EAC (Shaheen et al 2016).

Established factors for increased risk of BE and EAC include older age, male sex, white race, gastro-esophageal reflux, smoking and obesity, whereas gastric H. pylori infection is associated with a decreased risk (Spechler et al 2011). BE affects about 3.3 million adults in the U.S. (Ronkainen et al 2005), who are subjected to repeat endoscopies with biopsy over many years (Shaheen et al 2016), aiming to detect dysplasia and cancer at early stages. Overall, the presence of BE is associated with a 10-40 fold increased risk for the development of EAC (Sikkema et al 2010; Hvid-Jensen et al 2011); however, the overall rate of progression from non-dysplastic BE to EAC is only 0.1-0.5% per patient year, and recent analyses showed a pooled annual incidence of EAC in BE patients of 0.33% (Sikkema et al 2010; Hvid-Jensen et al 2011; Buas et al 2016; Desai et al 2012). Reported rates of progression from LGD to HGD/EAC are variable, ranging from 0.6% to 13.4% per year (Wani et al 2009). When HGD develops in BE, the risk for development of EAC greatly increases to 6%-19% per patient-year (Wani et al 2009). Guidelines for prevention of EAC recommend repeat surveillance endoscopies with multiple 4-quadrant biopsies every 1 to 2 cm along the length of the Barrett's mucosa, followed by pathological examination to detect BIM and dysplasia (Shaheen et al 2016; Caygill et al 2011).

Most patients on surveillance have Barrett's intestinal metaplasia negative for dysplasia, described as BIM throughout our study. Since only a small number of patients in the large BIM population undergoing endoscopic surveillance will develop dysplasia and/or EAC, molecular markers to identify BIM tissues that are likely to progress are warranted. We hypothesized that molecular alterations associated with risk of progression may occur in non-dysplastic BIM, and posited that identifying “molecular dysplasia” may be important to improve clinical management of BE and EAC patients.

There is considerable knowledge of the genomic and transcriptomic alterations that characterize EAC, with several large-scale genomic studies reported recently (Asan et al 2017; Dulak et al 2013; Secrier et al 2016). In recent years, a number of studies reported a spectrum of genomic alterations of Barrett's esophagus and pre-cancer dysplastic lesions (Del Portillo et al 2015; Agrawal et al 2012; Weaver et al 2014; Ross-Innes et al 2015; Stachler et al 2015). However, these molecular studies have been primarily cross-sectional with limited data reported from longitudinal cohorts (Li et al 2014). The difficulty in obtaining large longitudinal cohorts of progressors with non-dysplastic BIM available for testing before dysplasia/EAC development is well known to the Barrett's research community. Furthermore, it is recognized that there is limited knowledge of genomic alterations that may be more prevalent in early pre-progression-BIM, which could help identify dysplasia/EAC progressors, requiring additional data from longitudinal studies. Another limitation in published studies is that although patients received a diagnosis of BE/BIM and endoscopically were deemed to be negative for EAC, the biopsy fragments used for genomic assays were separate from the tissues characterized by histology, and they could in fact contain foci of dysplasia. The recent progress in genome-wide techniques to efficiently and accurately test formalin fixed paraffin embedded (FFPE) tissues makes it possible to test biopsy fragments that are histologically well-characterized to exclude the diagnosis of dysplasia/EAC before genomic assays are performed (Del Portillo et al 2015; Foster et al 2015).

Accordingly, there is a need for a more reliable diagnostic tool for early disease detection, which can be used to classify patients with BE into subgroups of low or high-risk of progressing to dysplasia or EAC, thereby has the potential to greatly improve surveillance and outcome of EAC. This invention is directed to meet these and other needs.

### SUMMARY OF THE INVENTION

Without being bound to a particular theory, the present invention is directed to characterize the landscape of somatic copy number alterations (SCNA) and mutations in a longitudinal cohort of BE patients undergoing surveillance, with a diagnosis of intestinal metaplasia negative for dysplasia, who went on to develop dysplasia and/or EAC, compared to a longitudinal cohort who did not develop dysplasia/EAC during the follow-up period. Further, the present invention utilizes clinical FFPE tissue samples obtained through endoscopic procedures and uses high-definition and high-sensitivity detection single nucleotide polymorphism (SNP) arrays and targeted next generation sequencing (NGS) for identification of early alterations that may represent genomic biomarkers of high-risk BIM negative for dysplasia that characterize progressors to dysplasia and/or EAC.

Accordingly, one aspect of the present invention is a method of predicting the risk of a subject having Barrett's Esophagus to develop a more severe condition. This method comprises the steps of:
a) obtaining a sample from the subject;
b) extracting DNA from the sample;
c) analyzing the DNA to detect a genomic alteration;
d) if the genomic alteration is detected, the subject is at high risk of developing a more severe condition; and
e) if the subject is determined to be at high risk, increasing the frequency of the subject's clinical screening.

Another aspect of the present invention is a method of predicting the risk of a subject having Barrett's Esophagus to develop dysplasia and/or esophageal adenocarcinoma (EAC). This method comprises the steps of:
a) obtaining a formalin fixed paraffin embedded (FFPE) biopsy sample from the subject;
b) extracting DNA from the sample;
c) analyzing the DNA to detect a genomic alteration, wherein the genomic alteration is somatic copy number alterations (SCNAs) in FHIT exon 5 and CDKN2A/2B;
d) if the genomic alteration is detected, the subject is at high risk of developing dysplasia and/or EAC; and
e) if the subject is determined to be at high risk, increasing the frequency of the subject's clinical screening.

Another aspect of the present invention is a method of supporting the diagnosis of dysplasia or esophageal adenocarcinoma (EAC) in a subject having Barrett's Esophagus. This method comprises the following steps:
a) obtaining a sample from the subject;
b) extracting DNA from the sample;
c) analyzing the DNA by both genome-wide single nucleotide polymorphism (SNP) arrays and next generation sequencing (NGS)to detect a genomic alteration;
d) if the genomic alteration is detected, the subject is likely to have dysplasia or EAC; and
e) treating the diagnosed subject for dysplasia or EAC.

Another aspect of the present invention is a method of supporting the diagnosis of dysplasia or esophageal adenocarcinoma (EAC) in a subject having Barrett's Esophagus. This method comprises the following steps:
a) obtaining a formalin fixed paraffin embedded (FFPE) biopsy sample from the subject;
b) extracting DNA from the sample;
c) analyzing the DNA by both genome-wide single nucleotide polymorphism (SNP) arrays and next generation sequencing (NGS) to detect somatic copy number alteration (SCNA) in TP53;
d) if a deletion of TP53 with loss of heterozygosity (LOH) is detected, the subject is likely to have dysplasia or EAC; and
e) treating the subject diagnosed with dysplasia or EAC with RF ablasion therapy.

A further aspect of the present invention is a kit for identifying a subject who has Barrett's Esophagus who is at high risk of progressing to dysplasia or esophageal adenocarcinoma (EAC). This kit comprises:
a) reagents for extracting DNA from a sample obtained from the subject;
b) a Barrett's Esophagus Progression to Cancer gene panel; and
c) instructions on how to stratify the subject into subgroups at low- or high-risk for progressing from Barrett's Esophagus to dysplasia or esophageal adenocarcinoma (EAC).

An additional aspect of the present invention is a kit for carrying out any of the aforementioned methods.

### BRIEF DESCRIPTION OF THE DRAWINGS

The application file contains at least one photograph executed in color. Copies of this patent application with color photographs will be provided by the Office upon request and payment of the necessary fee.

FIG. 1A shows the somatic copy number alterations (SCNAs) in samples tested by Oncoscan SNP arrays. NDBE: BIM of patients who never progressed to dysplasia or EAC. PP-BIM pre-progression biopsy samples of intestinal metaplasia negative for dysplasia at time point before dysplasia/EAC biopsy diagnosis in patient who progressed to dysplasia or EAC. C-BIM: Non-dysplastic intestinal metaplasia spatially separate from co-existing dysplasia or esophageal adenocarcinoma sampled at the same time point, representing concurrent intestinal metaplasia in patients with dysplasia/EAC. DAC: tissue lesion containing dysplasia or adenocarcinoma from progressor patients. Normal tissue: 6 patients who were tested for intestinal metaplasia also had matched non-esophageal normal tissues tested. Significance for comparisons of each lesion type with NDBE is indicated as: *p<0.5, **p<0.01, #p<0.001.

FIG. 1B shows the pathogenic somatic single nucleotide variants (SNVs) in samples tested by NGS targeted panels AmpliSeq and TruSeq. Significance for comparisons of each lesion type with NDBE is indicated as: *p<0.5, **p<0.01, #p<0.001.

FIG. 2A shows the distribution of genomic alterations in BIM of non-progressor (NDBE) samples.

FIG. 2B shows the distribution of genomic alterations in pre-progression BIM samples.

FIG. 2C shows the distribution of genomic alterations in concurrent BIM samples.

FIG. 2D shows the distribution of genomic alterations in low-grade dysplasia (LGD), high-grade-dysplasia (HGD) and adenocarcinoma (EAC) samples.

In FIGS. 2A to 2D, rows represent genes and columns represent patients. Genomic alterations are color coded: copy-neutral LOH (yellow), deletions (red), gains (blue), and pathogenic single nucleotide variants (SNV, green). The bar graphs on the right side of the plots represent the % of patients with alterations in each gene indicated on the left, and the bar graphs on the top represent the % of genomic alterations for each patient.

FIG. 3 shows the genomic alterations of multiple temporally and spatially separate samples for patient 7. Weighted log 2 ratio and B-allele frequency (BAF) plots were obtained with ChAS software. The left panel of FIG. 3 shows the region of chromosome 9p21.3-21.1 with the vertical interrupted line located over CDKN2A. The right panel of FIG. 3 shows the region of chromosome 3 including FHIT gene, with the vertical line over exon 5. Four samples from patient 7 are represented, including normal non-esophageal tissue, pre-progression-BIM 3 years and four months before progression (PP-BIM), concurrent-BIM (C-BIM), and EAC. The left panel of FIG. 3 plots show deletion of a region of chromosome 9p including CDKN2A in pre-progression-BIM (PP-BIM), concurrent-BIM (C-BIM), and EAC. In the pre-progression-BIM sample, this region comprises two hemizygous deletions highlighted by the “bubbles” in the BAF plot, and a homozygous deletion of CDKN2A, marked by lower log 2 ratio and loss of LOH with recovery of the middle line in the BAF plot due to the presence of normal cells in the sample. The EAC samples shows extension of the homozygous deletion encompassing CDKN2A and somatic copy neutral duplication of an entire 9p arm, as shown by the 4 lines in the BAF plot. The concurrent-BIM lesion is similar to the EAC, but the observed alterations are less prominent, probably due to much lower percentage of affected cells. The right panel of FIG. 3 plots show homozygous deletion of exon 5 of FHIT in the pre-progression-BIM, concurrent-BIM and EAC samples, and additionally, copy neutral partial LOH (CNpLOH) of chromosome 3 in EAC. Loss of CNpLOH in the region of FHIT deletion is likely due to the presence of normal cells with intact FHIT in the sample.

FIG. 4 shows the temporal sampling of patients. The x-axis shows the interval (in years) relative to the first sample. Patients are represented by rows and are grouped as patients who had pre-progression-baseline BIM (PP-BIM) samples tested, never dysplastic Barrett's esophagus patients (NDBE), and patients from whom only the concurrent BIM (C-BIM) and/or dysplasia or EAC (DAC) were tested. Each sample is represented by a rectangle with the color indicating the worst lesion in the sample (see legend). Samples tested by Oncoscan SNP-arrays are represented by a black border, and samples tested by NGS by a cross.

FIG. 5A to 5C show the whole-genome frequency plots of copy number alterations. Chromosomes are represented in the x-axis and the frequency of alterations in the y-axis. FIG. 5A shows the frequency of somatic gains and losses. Somatic gains are shown in red above the zero line and losses are shown in blue below the zero line. Representative genes in each frequency peak are summarized on the bottom graph, which displays the SCNAs in EAC lesions. FIG. 5B shows the frequency of somatic copy neutral loss of heterozygosity (LOH). FIG. 5C shows the frequency of other copy number alterations (i.e., gains and/or losses) likely present in the germ line.

FIG. 6 is a detailed view of somatic copy number alterations involving CDKN2A and FHIT. Each row represents an individual sample grouped by histopathological category (normal tissue, non-dysplastic BIM from NDBE patients, pre-progression-BIM [PP-BIM] of patients who progressed to LGD, HGD, or EAC, BIM concurrent with LGD, HGD, or EAC [C-BIM], and LGD, HGD or EAC samples). Each probe location in the array is represented by a small vertical line, and its color represents the individual probe log 2 ratio, from dark green (low) to red (high). Contiguous deleted segments are represented by an horizontal green line and contiguous areas of LOH by an horizontal blue line. The vertical arrows on the FHIT plot mark the location of exon 5. The vertical dark orange lines in the chromosome 9 plots represent the boundaries of CDKN2A and the light orange lines mark CDKN2B.

FIG. 7 shows the recurrent genomic alterations comparing pre-progression-BIM and subsequent dysplasia or EAC. All samples available in the database and obtained over time from each patient are represented: green—normal squamous mucosa; blue—non-dysplastic pre-progression BIM; orange—LGD; pink—HGD; and cells with red border—EAC. Rows represent patients and columns represent years since the pre-progression BIM sample was tested. Cells with black or red borders represent samples tested by both NGS and Oncoscan arrays and cells without borders represent non-tested samples. ND: no alterations detected. Downward arrows represent deletions, upward arrows represent gains, and the asterisks represent a deleterious SNV. In patient 4, the same TP53 mutation found in HGD was present in pre-progression-BIM at 3% variant allele frequency, below our threshold for calling single nucleotide variants.

### DETAILED DESCRIPTION OF THE INVENTION

Genomic alterations detectable in tissue samples obtained during surveillance of Barrett's esophagus patients may serve as biomarkers for risk stratification to improve detection of progressors to dysplasia and EAC. A number of recent studies reported a spectrum of genomic alterations in non-dysplastic BE and in dysplastic pre-cancer and EAC lesions (Del Portillo et al 2015; Agrawal et al 2012; Weaver et al 2014; Ross-Innes et al 2015; Stachler et al 2015). However, most studies were cross-sectional (Del Portillo et al 2015; Agrawal et al 2012; Ross-Innes et al 2015; Stachler et al 2015) and there are limited data from pre-progression longitudinal samples reporting genome-wide alterations in BIM (Li et al 2014).

In the present invention, a longitudinal study of Barrett's esophagus progression from BIM samples characterized histologically as negative for dysplasia was carried out for the first time by performing SNP arrays and NGS from FFPE tissue samples, the results of which showed that genomic alterations primarily in FHIT exon 5 and CDKN2A/B are frequently detected in routine FFPE biopsy samples of non-dysplastic Barrett's epithelium of patients who harbor or develop future dysplasia or EAC, suggesting they represent biomarkers of progression that may be incorporated in routine workup for cancer surveillance in Barrett's esophagus patients.

Accordingly, one aspect of the present invention is a method of predicting the risk of a subject having Barrett's Esophagus to develop a more severe condition. This method comprises the steps of:
a) obtaining a sample from the subject;
b) extracting DNA from the sample;
c) analyzing the DNA to detect a genomic alteration;
d) if the genomic alteration is detected, the subject is at high risk of developing a more severe condition; and
e) if the subject is determined to be at high risk, increasing the frequency of the subject's clinical screening.

In some embodiment of this aspect, the analysis of DNA in step c) is performed with a Barrett's Esophagus Progression to Cancer (BPC) gene panel comprising the following genes: APC, CDKN2A, CDKN2B, FHIT and TP53.

In some embodiment of this aspect, the more severe condition is selected from low-grade dysplasia (LGD), high-grade dysplasia (HGD) and esophageal adenocarcinoma (EAC). As used herein, “esophageal adenocarcinoma (EAC)” includes intramucosal adenocarcinomas, superficial submucosal adenocarcinomas and invasive adenocarcinomas.

In some embodiments of this aspect, the sample can be obtained from Barrett's tissue, cytology preparations, circulating cells, or blood.

In some embodiments of this aspect, the sample can be fresh sample or formalin fixed paraffin embedded (FFPE) sample. Preferably, the sample is FFPE sample.

As used herein, “genomic alteration” can refer to the accumulation of extra copies of DNA or chromosomes, chromosomal translocations, chromosomal inversions, chromosome deletions, single-strand breaks in DNA, double-strand breaks in DNA, the intercalation of foreign substances into the DNA double helix, or any abnormal changes in DNA tertiary structure that can cause either the loss of DNA, or the misexpression of genes. In some embodiments, the genomic alteration may include single nucleotide variants, complex insertions and deletions, genomic losses and gains, genome copy number changes, copy-neutral loss of heterozygosity, and combinations thereof.

As used herein, a “single-nucleotide variant (SNV)” is a variation in a single nucleotide without any limitations of frequency and may arise in somatic cells. A somatic single-nucleotide variation (e.g., caused by cancer) may also be called a single-nucleotide alteration.

In certain embodiments of this aspect, the genomic alteration is a somatic copy number alteration (SCNA), which can be selected from the group consisting of deletion of FHIT including exon 5, hem izygous deletion of CDKN2A/2B with partial loss of heterozygosity (pLOH), other somatic deletions, and combinations thereof. The other somatic deletions, in particular, may include hemizygous deletion of PRIM2 with pLOH, hemizygous deletion of chromosome 9p21.3-21.1 with pLOH, deletion of APC without pLOH, deletion of OVOS without pLOH, deletion of STYX without pLOH, deletion of DCC without pLOH, deletion of PLCB1 without pLOH, multiple deletions of MTOR, chromosome 5q22.2, chromosome 9p13.3, OVOS, SPRED1, SMAD7, PLCB1 and

PLA2G3, and combinations thereof.

As used herein, “somatic copy number alteration (SCNA)” refers to somatic changes of chromosome structure that result in gain or loss in copies of sections of DNA.

As used herein, an “allele” is a variant form of a given gene. If both alleles of a diploid organism are the same, the organism is “homozygous” at that locus. If they are different, the organism is “heterozygous” at that locus. If one allele is missing, it is “hemizygous”, and, if both alleles are missing, it is “nullizygous”. As used herein, “loss of heterozygosity (LOH)” is a cross chromosomal event that results in loss of the entire gene and the surrounding chromosomal region. The term “copy-neutral loss of heterozygosity” means no net change in the copy number occurs in the affected individual. Possible causes for copy-neutral LOH include acquired uniparental disomy (UPD) and gene conversion. In UPD, a person receives two copies of a chromosome, or part of a chromosome, from one parent and no copies from the other parent due to errors in meiosis I or meiosis II. This acquired homozygosity could lead to development of cancer if the individual inherited a non-functional allele of a tumor suppressor gene.

In certain embodiments of this aspect, the genomic alteration is a pathogenic mutation. More preferably, such pathogenic mutation is a TP53 mutation.

In some embodiments of this aspect, the somatic copy number alteration (SCNA) is detected by genome-wide single nucleotide polymorphism (SNP) arrays. In some embodiments of this aspect, the pathogenic mutation is detected by targeted next generation sequencing (NGS) with a cancer panel.

As used herein, a “single-nucleotide polymorphism”, often abbreviated to “SNP”, is a variation in a single nucleotide that occurs at a specific position in the genome, where each variation is present to some appreciable degree within a population (e.g. >1%). SNPs underlie differences in a subject's susceptibility to a disease, and may also indicate the severity of illness as well as the way the subject's body responds to treatments. The “single nucleotide polymorphism (SNP) arrays” is a type of DNA microarray which is used to detect polymorphisms within a population. An SNP arrays comprises three mandatory components: 1) an array containing immobilized allele-specific oligonucleotide (ASO) probes; 2) fragmented nucleic acid sequences of target, labelled with fluorescent dyes; 3) a detection system that records and interprets the hybridization signal.

As used herein, “next-generation sequencing (NGS)” refers to a non-Sanger-based high-throughput methodology that enables rapid sequencing of the base pairs in DNA or RNA samples. NGS supports a broad range of applications, including gene expression profiling, chromosome counting, detection of epigenetic changes, and molecular analysis. Common NGSs include but are not limited to, for example, Massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, Single molecule real time (SMRT) sequencing, Nanopore DNA sequencing.

As used herein, a “cancer panel” refers to a technology that simultaneously examines a number of different genes to look for potentially cancer-causing mutations, which can provide information to help people take action to prevent or stop cancer. In certain embodiments, the cancer panel is a TruSeq cancer panel comprising the following 48 genes: ABL1, AKT1, ALK, APC, ATM, BRAF, CDH1, CDKN2A, CSF1R, CTNNB1, EGFR, ERBB2, ERBB4, FBXW7, FGFR1, FGFR2, FGFR3, FLT3, GNA11, GNAS, GNAQ, HNF1A, HRAS, JAK2, JAK3, IDH1, KDR/VEGFR2, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, and VHL. In certain embodiments, the cancer panel is an AmpliSeq cancer panel comprising the following 50 genes: ABL1, AKT1, ALK, APC, ATM, BRAF, CDH1, CDKN2A, CSF1R, CTNNB1, EGFR, ERBB2, ERBB4, EZH2, FBXW7, FGFR1, FGFR2, FGFR3, FLT3, GNA11, GNAS, GNAQ, HNF1A, HRAS, JAK2, JAK3, IDH1, IDH2, KDR/VEGFR2, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, and VHL.

Another aspect of the present invention is a method of predicting the risk of a subject having Barrett's Esophagus to develop dysplasia and/or esophageal adenocarcinoma (EAC). This method comprises the steps of:
a) obtaining a formalin fixed paraffin embedded (FFPE) biopsy sample from the subject;
b) extracting DNA from the sample;
c) analyzing the DNA to detect a genomic alteration, wherein the genomic alteration is somatic copy number alterations (SCNAs) in FHIT exon 5 and CDKN2A/2B;
d) if the genomic alteration is detected, the subject is at high risk of developing dysplasia and/or EAC; and
e) if the subject is determined to be at high risk, increasing the frequency of the subject's clinical screening.

In some embodiments of this aspect, the analysis of DNA in step c) is performed with a Barrett's Esophagus Progression to Cancer (BPC) gene panel comprising the following genes: APC, CDKN2A, CDKN2B, FHIT and TP53.

In some embodiments of this aspect, the genomic alteration is a combination of SCNAs in FHIT exon 5 and CDKN2A/2B plus a TP53 mutation.

Another aspect of the present invention is a method of supporting the diagnosis of dysplasia or esophageal adenocarcinoma (EAC) in a subject having Barrett's Esophagus. This method comprises the following steps:
a) obtaining a sample from the subject;
b) extracting DNA from the sample;
c) analyzing the DNA by both genome-wide single nucleotide polymorphism (SNP) arrays and next generation sequencing (NGS)to detect a genomic alteration;
d) if the genomic alteration is detected, the subject is likely to have dysplasia or EAC; and
e) treating the diagnosed subject for dysplasia or EAC.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, a “subject” is a mammal, preferably, a human.

In some embodiments of this aspcect, the analysis of DNA in step c) is performed with a Barrett's Esophagus Progression to Cancer (BPC) gene panel comprising the following genes: APC, CDKN2A, CDKN2B, FHIT and TP53.

In certain embodiments of this aspect, the genomic alteration is a somatic copy number alteration (SCNA), and in particular, a deletion of TP53 with loss of heterozygosity (LOH).

Yet another aspect of the present invention is a method of supporting the diagnosis of dysplasia or esophageal adenocarcinoma (EAC) in a subject having Barrett's Esophagus. This method comprises the following steps:
a) obtaining a formalin fixed paraffin embedded (FFPE) biopsy sample from the subject;
b) extracting DNA from the sample;
c) analyzing the DNA by both genome-wide single nucleotide polymorphism (SNP) arrays and next generation sequencing (NGS) to detect somatic copy number alteration (SCNA) in TP53;
d) if a deletion of TP53 with loss of heterozygosity (LOH) is detected, the subject is likely to have dysplasia or EAC; and
e) treating the subject diagnosed with dysplasia or EAC with RF ablasion therapy.

As used herein, “ablation therapy” refers to a type of minimally invasive procedure doctors use to destroy abnormal tissue that occurs with many conditions. Non-limiting types of ablation include: atrial fibrillation ablation, cardiac ablation, cryoablation for cancer, laser PVP surgery, radiofrequency (RF) ablation for cancer and transurethral needle ablation (TUNA). Radiofrequency (RF) ablation therapy for cancer is a minimally invasive procedure that uses electrical energy and heat to destroy cancer cells in, for example, adrenal gland, breast, bone, kidney, liver, lung, pancreas, thyroid. Radiofrequency (RF) ablation is also an option for treating precancerous cells in the esophagus that are associated with Barrett's esophagus.

In some embodiments of this aspect, the analysis of DNA in step c) is performed with a Barrett's Esophagus Progression to Cancer (BPC) gene panel comprising the following genes: APC, CDKN2A, CDKN2B, FHIT and TP53.

A further aspect of the present invention is a kit for identifying a subject who has Barrett's Esophagus who is at high risk of progressing to dysplasia or esophageal adenocarcinoma (EAC). This kit comprises:
a) reagents for extracting DNA from a sample obtained from the subject;
b) a Barrett's Esophagus Progression to Cancer gene panel; and
c) instructions on how to stratify the subject into subgroups at low- or high-risk for progressing from Barrett's Esophagus to dysplasia or esophageal adenocarcinoma (EAC).

In some embodiments of this aspect, the Barrett's Esophagus Progression to Cancer gene panel comprises the following genes: APC, CDKN2A, CDKN2B, FHIT and TP53.

The kits may also include suitable storage containers, e.g., ampules, vials, tubes, etc., for each compound of the present invention (which, e.g., may be in the form of pharmaceutical compositions) and other reagents, e.g., buffers, balanced salt solutions, etc., for use in administering the active agents to subjects. The compounds and/or pharmaceutical compositions of the invention and other reagents may be present in the kits in any convenient form, such as, e.g., in a solution or in a powder form. The kits may further include a packaging container, optionally having one or more partitions for housing the compounds and/or pharmaceutical compositions and other optional reagents.

Still another aspect of the present invention is a kit for carrying out any of the methods disclosed above.

### EXAMPLES

The invention is further illustrated by the following examples, which are offered for illustrative purposes, and are not intended to limit the invention in any manner. Those of skill in the art will readily recognize a variety of noncritical parameters, which can be changed or modified to yield essentially the same results.

### Example 1

### Methods and Materials

### Patients and Sample Characteristics

The primary objective of this study was to identify patients with longitudinal samples of BE, before progression to dysplasia or adenocarcinoma, in order to identify genomic alterations that characterize BIM tissues of patients who develop future dysplasia or adenocarcinoma. One specimen was tested for each patient, from FFPE-embedded tissue blocks generally including multiple biopsy fragments containing intestinal metaplasia (BIM), or from representative areas of BIM microdissected from EMR or surgical resection specimens.

Sixty-three patients were included in the study, divided into four groups (see Table 1):

Group 1: Eighteen progressor patients (pre-progression-BIM) with a mean age of 61.5 years (range 43-77, standard error of the mean [SEM]=2.2), 14 male and 4 female, were included in this group. The median time of surveillance from the tested pre-progression-BIM biopsy sample to first diagnosis of dysplasia or adenocarcinoma was 43 months (range 12 to 134, SEM=7.5); 6 patients progressed to high-grade dysplasia (HGD), 5 to low-grade dysplasia (LGD) and 7 to adenocarcinoma (EAC), including intramucosal adenocarcinomas (pT1a), a superficial submucosal adenocarcinoma (T1b) and invasive adenocarcinomas.

Group 2: Twenty-seven patients with a mean age of 67.0 (range 42-85, SEM=1.8), 20 male and 7 female, who had a histological diagnosis of BIM, negative for dysplasia, in at least two biopsies within over 2 consecutive years (median 51 months, range 25-114) and never had a diagnosis of dysplasia (HGD or LGD) or EAC in any esophageal biopsy or resection, represented the non-progressor, never dysplastic Barrett's esophagus (NDBE) group.

The total known duration of BE for the pre-progression-BIM group (first sample available in the database to the first sample with dysplasia/EAC had a median of 45 months (range 12-143, SEM=10.5). The total known duration of BE in the NDBE group (first sample available to last sample available) had a median of 82 months (range 25-153, SEM=6.7).

The mean length of Barrett's in pre-progression-BIM was 5.0 cm (range 1-13, SEM=0.9) with 1 case described as BIM in GE junction and for NDBE was 5.0 cm (range 3-14 cm, SEM=0.7) with 4 cases described as BIM in GE junction.

There were no statistically significant differences between pre-progression-BIM and NDBE patients in age, length of BE, or duration of BE.

Group 3: Fifteen patients with dysplasia or EAC who had temporally concurrent but spatially separate BIM samples from the same procedure (endoscopic, EMR, or surgical resection). These patients did not have baseline pre-progression biopsies available. In this group the esophageal mucosa with BIM negative for dysplasia, was described as concurrent-BIM and in some cases the matched dysplasia or EAC lesions were tested.

Group 4: Three patients who had only dysplasia or EAC lesions tested.

Descriptive statistics for Groups 3 and 4 combined are shown on Table 1, with no statistically differences in age (median 65, range 40-83, SEM=2.5), sex distribution (72% male), or length of BE (median 5.0, range 3-17, SEM=1.0), as compared to Group 1 (progressors) or Group 2 (NDBE).

Six matched concurrent-BIM and 10 HGD/EAC samples from Group 1 were tested at the time of dysplasia/EAC diagnosis, resulting in a total of 21 patients with concurrent-BIM tested and 13 patients with dysplasia/EAC lesions tested. Most of the tested EACs were intramucosal (T1a) (see Table 1).

Most patients were identified in the Columbia Barrett's Pathology and Archival Database, which includes 32,725 patients with FFPE esophageal specimens from January 2000 to 2017. The study was approved by the institutional review board of Columbia University.

TABLE 1
Patients and specimen characteristics.

| | | | | | Length | BIM | Pre- | | | | | | |
|---|---|---|---|---|---|---|---|---|---|---|---|---|---|
| | | | | Sample | BE | Area | Sample | Interval | Worse Pt | | SNP | C-BIM | DAC |
| Group | Patient | Age | Sex | Type | (cm) | (%) | (Months) | (Months) | Diagnosis | NGS | Array | Tested | Tested |
| PP-BIM | 1 | 55 | F | bx | 7 | 15% | 0 | 25 | EAC (T1a) | TS | Y | N | Y |
| PP-BIM | 2 | 70 | M | bx | 3 | 90% | 2 | 28 | EAC (T1a) | TS | Y | Y | Y |
| PP-BIM | 3 | 77 | F | bx | 3 | 1% | 0 | 74 | HGD | AS | Y | N | Y |
| PP-BIM | 4 | 61 | M | bx | 4 | 20% | 6 | 14 | HGD | TS | Y | Y | Y |
| PP-BIM | 5 * | 65 | M | bx | GEJ | 10% | 0 | 134 | EAC (T3) | AS | Y | N | Y |
| PP-BIM | 6 | 61 | M | bx | 5 | 50% | 0 | 21 | LGD | TS | Y | Y | Y |
| PP-BIM | 7 * | 70 | F | bx | 5 | 30% | 3 | 40 | EAC (T1a) | TS | Y | Y | Y |
| PP-BIM | 8 | 62 | M | bx | 5 | 20% | 67 | 76 | EAC (T1b) | TS | Y | Y | Y |
| PP-BIM | 9 | 74 | M | bx | 4 | 30% | 0 | 48 | HGD | TS | Y | N | Y |
| PP-BIM | 10 | 43 | M | bx | 11 | 80% | 0 | 12 | LGD | AS | Y | N | N |
| PP-BIM | 11 @ | 43 | M | bx | 4 | 70% | 23 | 68 | LGD | AS | Y | N | N |
| PP-BIM | 12 @ | 57 | M | bx | 1 | 80% | 0 | 23 | HGD | AS | Y | N | N |
| PP-BIM | 13 # | 75 | M | bx | 13 | 90% | 0 | 45 | EAC | AS | Y | N | N |
| PP-BIM | 14 # | 55 | M | bx | 13 | 80% | 0 | 79 | EAC | AS | Y | N | N |
| PP-BIM | 15 @ | 68 | M | bx | 5 | 60% | 0 | 45 | LGD | N | Y | N | N |
| PP-BIM | 16 | 64 | F | bx | 7 | 90% | 128 | 15 | HGD | N | Y | N | N |
| PP-BIM | 17 * | 57 | M | bx | 10 | 25% | 29 | 66 | LGD | AS | N | Y | Y |
| PP-BIM | 18 | 60 | M | bx | Unknown | 40% | 0 | 14 | HGD | AS | N | N | N |
| TOTAL | N = 18 | 61.5 | 78% M | 100% bx | 5.0 | 45% | 0 | 43 | 28% LGD, | 39% TS | 89% Y | 33% Y | 56% Y |
| PP-BIM | | (43-77) | | | (1-13) | (1-90) | (0-128) | (12-134) | 33% HGD, | 50% AS | | | |
| | | | | | | | | | 39% EAC | | | | |
| NDBE | 19 | 55 | F | bx | 14 | 40% | 37 | 93 | BIM | TS | Y | NA | NA |
| NDBE | 20 | 54 | F | bx | 3 | 50% | 17 | 70 | BIM | TS | Y | NA | NA |
| NDBE | 21 | 71 | M | bx | 3 | 20% | 38 | 53 | BIM | TS | Y | NA | NA |
| NDBE | 22 | 76 | F | bx | 5 | 20% | 31 | 48 | BIM | TS | Y | NA | NA |
| NDBE | 23 | 65 | M | bx | 13 | 10% | 0 | 82 | BIM | TS | Y | NA | NA |
| NDBE | 24 | 61 | M | bx | 4 | 50% | 23 | 48 | BIM | TS | Y | NA | NA |
| NDBE | 25 | 70 | F | bx | 6 | 30% | 0 | 114 | BIM | TS | Y | NA | NA |
| NDBE | 26 | 67 | M | bx | 3 | 80% | 9 | 51 | BIM | AS | Y | NA | NA |
| NDBE | 27 | 70 | F | bx | 6 | 70% | 118 | 35 | BIM | AS | Y | NA | NA |
| NDBE | 28 | 69 | M | bx | 3 | 20% | 44 | 50 | BIM | AS | Y | NA | NA |
| NDBE | 29 | 74 | M | bx | 5 | 15% | 16 | 96 | BIM | AS | Y | NA | NA |
| NDBE | 30 | 59 | M | bx | 3 | 20% | 42 | 40 | BIM | AS | Y | NA | NA |
| NDBE | 31 | 71 | M | bx | GEJ | 30% | 0 | 77 | BIM | AS | Y | NA | NA |
| NDBE | 32 | 74 | M | bx | 6 | 10% | 18 | 74 | BIM | AS | Y | NA | NA |
| NDBE | 33 | 55 | F | bx | GEJ | 60% | 0 | 45 | BIM | AS | Y | NA | NA |
| NDBE | 34 | 70 | M | bx | 3 | 5% | 20 | 49 | BIM | AS | Y | NA | NA |
| NDBE | 35 | 53 | M | bx | Unknown | 20% | 11 | 36 | BIM | AS | N | NA | NA |
| NDBE | 36 | 79 | M | bx | D3 | 70% | 27 | 43 | BIM | AS | Y | NA | NA |
| NDBE | 37 | 63 | M | bx | 8 | 5% | 57 | 58 | BIM | AS | N | NA | NA |
| NDBE | 38 | 68 | M | bx | 5 | 20% | 93 | 26 | BIM | AS | Y | NA | NA |
| NDBE | 39 | 42 | M | bx | GEJ | 20% | 61 | 31 | BIM | AS | Y | NA | NA |
| NDBE | 40 | 67 | M | bx | GEJ | 30% | 2 | 57 | BIM | AS | Y | NA | NA |
| NDBE | 41 | 76 | M | bx | D3 | 10% | 22 | 60 | BIM | AS | Y | NA | NA |
| NDBE | 42 | 64 | M | bx | 3 | 10% | 0 | 58 | BIM | AS | N | NA | NA |
| NDBE | 43 | 65 | M | bx | 3 | 50% | 0 | 25 | BIM | AS | N | NA | NA |
| NDBE | 44 | 85 | F | bx | 11 | 80% | 0 | 56 | BIM | TS | N | NA | NA |
| NDBE | 45 | 56 | M | bx | 13 | 65% | 31 | 31 | BIM | TS | N | NA | NA |
| TOTAL | N = 27 | 67.0 | 74% M | 108% bx | 5.0 | 20% | 20 | 51 | 180% BIM | 33% TS | 78% Y | NA | NA |
| NDBE | | (42-85) | | | (3-14) | (5-80) | (0-118) | (25-114) | | 67% AS | | | |
| C-BIM | 46 | 75 | F | bx | 17 | 90% | 2 | NA | EAC (T1a) | TS | Y | Y | N |
| C-BIM | 47 | 69 | M | bx | 6 | 10% | 0 | NA | HGD | N | Y | Y | N |
| C-BIM | 48 * | 64 | M | bx | 10 | 10% | 0 | NA | EAC (T1a) | TS | Y | Y | N |
| C-BIM | 49 | 65 | F | bx | 5 | 70% | 0 | NA | HGD | TS | Y | Y | N |
| C-BIM | 50 | 66 | M | bx | 6 | 70% | 6 | NA | EAC (T1a) | TS | Y | Y | N |
| C-BIM | 51 | 65 | M | bx | 3 | 10% | 0 | NA | HGD | TS | Y | Y | N |
| C-BIM | 52 | 56 | M | bx | 3 | 70% | 0 | NA | EAC (T1a) | TS | Y | Y | N |
| C-BIM | 53 | 40 | F | bx | 4 | 30% | 0 | NA | HGD | TS | Y | Y | N |
| C-BIM | 54 | 62 | M | bx | 5 | 60% | 1 | NA | EAC (T1a) | TS | Y | Y | N |
| C-BIM | 55 * | 71 | M | EMR | 3 | 30% | 7 | NA | EAC (T1a) | N | Y | Y | N |
| C-BIM | 56 | 57 | M | Res | 3 | 10% | 2 | NA | EAC (T1b) | AS | Y | Y | N |
| C-BIM | 57 | 65 | M | Res | 10 | 70% | 0 | NA | EAC (T2) | AS | Y | Y | N |
| C-BIM | 58 | 79 | M | Res | Unknown | 10% | 0 | NA | EAC (T3) | AS | Y | Y | N |
| C-BIM | 59 | 57 | M | bx | 6 | 20% | 0 | NA | EAC | AS | Y | Y | N |
| C-BIM | 60 | 71 | F | bx | 12 | 30% | 3 | NA | HGD | AS | Y | Y | N |
| DAC | 61 | 83 | F | EMR | Unknown | NA | 0 | NA | EAC (T1a) | AS | Y | N | Y |
| DAC | 62 | 47 | M | EMR | 5 | NA | 31 | NA | HGD | AS | Y | N | Y |
| DAC | 63 | 84 | M | EMR | Unknown | NA | 0 | NA | EAC (T1a) | AS | Y | N | Y |
| C-BIM/ | N = 18 | 65.0 | 72% | 61% bx | 5.0 | 30% | 0 | 36 | 33% HGD | 89% Y | 100% Y | 83% Y | 17% Y |
| DAC | | (40-83) | | 22% EMR | (3-17) | (10-90) | (0-31) | (8-75) | 67% EAC | | | | |
| Totals | | | | 17% Res | | | | | | | | | |

Note:
Sixty-three patients had SNP arrays and/or NGS targeted panels performed (AS: AmpliSeq, TS: TruSeq). Rows in bold after each group show total numbers of patients, median and range of numeric variables, or frequencies for categorical variables. Age: Patient age, in years, when first sample was tested. In the Sex column, M: male, F: female. In the Sample Type column, Bx: biopsy. EMR: endoscopic mucosal resection, Res: surgical resection (esophagectomy). Length BE shows the longitudinal length in cm of the endoscopically evident Barrett's mucosa. The BIM Area column shows the area of intestinal metaplasia as a percentage of the total sample tested, as assessed by histopathological examination of adjacent H&E slides. The Pre-Sample column is the interval in months between the first sample available in our database and the first tested sample. The Interval column is the interval in months between the first tested baseline Barrett's intestinal metaplasia of progressors (pre-progresion-BIM/PP-BIM) sample and the first sample with dysplasia or EAC in the PP-BIM group (representing the pre-progression interval), or the interval between the tested sample and the last sample available in the database in the never dysplastic non-progressors (NDBE), concurrent-BIM (C-BIM), and dysplasia and EAC (DAC) groups (representing the follow-up interval). In the NGS column, when possible to determine from the pathology report, the T stage is shown in parenthesis following EAC: stage T1a represents intramucosal adenocarcinoma, subsequent stages indicate invasion into the esophageal submucosa (T1b), muscularis propria (T2), adventitia (T3) and adjacent organs (T4). In the PP-BIM group, in addition to the pre-progression PP-BIM sample, some patients had subsequent samples with DAC or C-BIM tested by SNP arrays (see last 2 columns). In the last 3 columns, Y: Yes (tested). N: Not tested. NA: not applicable.
* Matching normal tissue also tested.
# contributed from the University of Pittsburgh by J D.
@ Contributed from the University of Pennsylvania by G W F.

### Tissue Microdissection

Hematoxylin and eosin (H&E) stained sections of the selected tissue blocks were assessed for presence, extent, and grade of lesions by two gastrointestinal pathologists (A.R.S. and S.K.). Lesional areas for microdissection were marked on the guiding H&E slides. The BIM samples used for DNA extraction had areas of intestinal metaplasia ranging from 1 to 90% (median: 45%) of the overall mucosa in pre-progression-BIM, 20% (range 5 to 80%) in NDBE samples and 30% (range 10 to 90%) in concurrent-BIM samples, with no significant differences between groups (Table 1), as estimated in the guiding H&E-stained sections. The mucosa background consisted of cardia, cardio-oxyntic, and squamous mucosa, as is usual in endoscopic biopsy samples of BE patients.

### DNA Extraction and Quantification

For DNA extraction we used unstained sections, 5 to 7 micron thick. Genomic DNA was isolated using QIAamp DNA FFPE Tissue Kit (Qiagen, Germantown, Md.) following the manufacturer recommendations. DNA was quantified by fluorimetry using Quant-iT dsDNA HS Assay (Invitrogen, Carlsbad, Calif.) and measured by the Qubit fluorimeter (Invitrogen).

### Oncoscan SNP Arrays

The DNA samples were processed for identification of somatic copy number alterations (SCNAs) using OncoScan FFPE Assay (Affymetrix, Santa Clara, Calif.) or OncoScan CNV Assay (Affymetrix); both utilize the Molecular Inversion Probe (MIP) assay technology. The annealing, amplification and labeling were performed as described in OncoScan FFPE Assay Manual Rev.1 (Affymetrix). Briefly, 79 ng of DNA from each sample were annealed with the MIP for 18 h followed by gap filling with AT/GC nucleotides. After removing the un-ligated, non-gap-filled, linear probes through exonuclease treatment, a cleavage enzyme was added to linearize the gap-filled circular MIP followed by amplification and enrichment of the gap-filled, linearized MIP through 1st and 2nd PCR. After fragmentation with HaeIII enzyme, the enriched PCR product was hybridized to the GeneChip Oncoscan Array (Affymetrix) or GeneChip Oncoscan CNV Array (Affymetrix) for 16-18h at 49° C. in a 60 rpm rotator. After hybridization, the arrays were washed, stained using GeneChip Fluidics Station 450 (Affymetrix) and scanned using GeneChip Scanner 3000 7G (Affymetrix). The CEL files generated after scanning were converted to OSCHP files and analyzed using Chromosome Analysis Suite 3.2 (ChAS) Software (Affymetrix).

The B-allelic frequency (BAF) and log 2-ratio plots were visually inspected using ChAS and segments were validated and manually adjusted. In addition, an automated algorithm using R statistical software counted the number of consecutive probes on the 5′ and 3′ regions around FHIT exon 5 with weighted log 2 ratios less than zero. FHIT exon 5 was considered deleted if there were five of more consecutive probes with weighted log 2-ratios <0 on each side of exon 5 and the minimum weighted log 2-ratio of those probes were <-0.5 on both sides of exon 5.

### Next Generation Sequencing (NGS)

Sixteen pre-progression-BIM and NDBE samples and 24 concurrent-BIM, dysplasia, and EAC samples were sequenced with the TruSeq Amplicon Cancer Panel (Illumina, San Diego, Calif.) (see Tables 1 and 8). Because of low DNA yield, we performed the more sensitive AmpliSeq Cancer Hotspot panel (Life Technologies, Carlsbad, Calif.) in 27 pre-progression-BIM and NDBE samples, and 8 concurrent-BIM, dysplasia, and EAC samples. It has been previously shown that the results from these two NGS panels were comparable in BIM samples (Del Portillo et al 2015).

AmpliSeq: The AmpliSeq Cancer Hotspot panel version 2 (Life Technologies, Carlsbad, Calif.) used in the study includes 207 amplicon target regions ranging from 111 to 187bp in size, covering 50 oncogenes and tumor suppressor genes previously implicated in cancer, and more than 2800 sequence variants described in the COSMIC database of cancer mutations. This gene panel covers the following cancer genes: ABL1, AKT1, ALK, APC, ATM, BRAF, CDH1, CDKN2A, CSF1R, CTNNB1, EGFR, ERBB2, ERBB4, EZH2, FBXW7, FGFR1, FGFR2, FGFR3, FLT3, GNA11, GNAS, GNAQ, HNF1A, HRAS, JAK2, JAK3, IDH1, IDH2, KDR/VEGFR2, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, and VHL.

Briefly, the primer pool was mixed with 10 to 15 ng of DNA and the AmpliSeq HiFi master mix (Life Technologies), and amplification was performed following the manufacturer instructions. Primers were partially digested with FuPa, Ion Express Barcode adapters were attached to the amplicons, and the library was purified using AmPure beads (Beckman and Coulter, Brea, Calif.). For quality assessment, the barcoded libraries were quantified on a Qubit fluorimeter with Picogreen (Life Technologies) and size was analyzed with BioAnalyzer (Agilent Technologies, Santa Clara, Calif.). Six libraries were pooled in equimolar ratios, diluted to 20 pM, and further amplified using the Ion OneTouch 200 system. Sequencing was performed on a 318 chip in the Ion PGM sequencer (Life Technologies).

Ion Torrent sequencing data were analyzed with the Ion Torrent Suite version 4.4 (Life Technologies). Bar-coded reads were deconvoluted and aligned to the human reference genome build 37 (hg19) using TMAP torrent mapping alignment program. The Ion Torrent's Variant Caller was used to analyze resulting .bam files to detect single nucleotide variants and indels.

TruSeq: We used the TruSeq Amplicon Cancer Panel and performed sequencing with the MiSeq reagent kit v2 (Illumina, San Diego, Calif.), according to the manufacturer instructions. Briefly, we used 250 ng of genomic DNA obtained from FFPE tissue in sequencing reactions run on a paired end 2×150 bp cycler run. The TruSeq Amplicon Cancer Panel targets mutational hotspots in 48 genes implicated in cancer, including all but the EZH2 and IDH2 genes, as compared to the AmpliSeq panel. MiSeq raw sequencing files were processed with MiSeq Reporter version 2.6 software (Illumina Inc. Somatic Variant Caller. http://res.illumina.com/documents/products/technotes/technote somatic_variant_caller. pdf). The output VCF files contain the identified variants and associated quality (Q) scores.

VCF files resulting from the Illumina or IonTorrent analysis pipelines were annotated using the Annovar tool version 2016Feb01 (http://www.openbioinformatics.org/annovar) and non-synonymous genomic variants were rejected if they fulfilled any of the following criteria:
1) Base calls <Q20
2) Depth of coverage <40
3) Strand bias <−3.0
4) Variants within less than 5 bp of the amplicon edge or in regions of homopolymers: variant allelic frequency (VAF) <20%; remaining variants: VAF <5%
5) Allelic frequency in any human population >2%

Non-synonymous variants were inspected using the Integrated Genome Viewer software version 2.3.7 (Thorvaldsdottir et al 2013).

### Statistical Analyses

The Pearson Chi-square test was used to identify differences in the frequency of genomic alterations between groups (e.g. pre-progression-BIM vs. NDBE). Two-tailed t-tests were used to determine whether there were significant age, length of BE, area of lesional tissue, or size of non-diploid genome differences between groups. R Statistical Software was used. P values <0.05 were considered significant.

### Example 2

### Somatic Genomic Copy Number Alterations in Pre-Progression Non-Dysplastic Barrett's Intestinal Metaplasia are Highly Prevalent in Patients Who Progress to Dysplasia and Adenocarcinoma

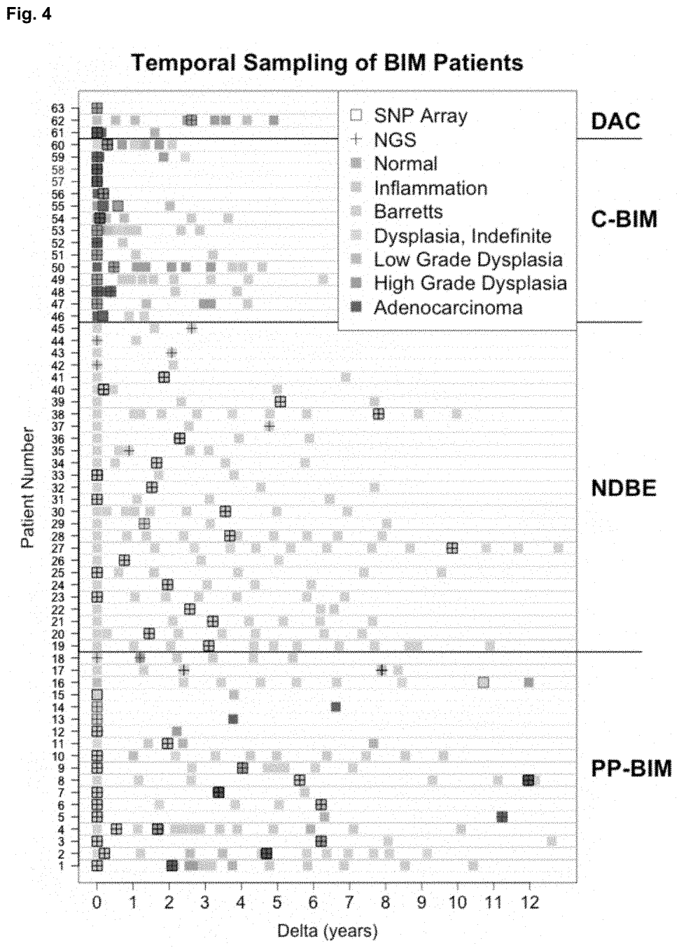

Most BE patients who are on endoscopic surveillance have BIM, negative for dysplasia, and only a small subset of these patients will progress to dysplasia/EAC. Genomic characterization, beyond histopathology, of BIM samples that are negative for dysplasia may be useful to separate progressors from non-progressors to better tailor endoscopic surveillance and therapy. Therefore, we identified a group of patients with FFPE endoscopic biopsies obtained before the development of dysplasia/EAC (described as baseline BIM, pre-progression-BIM), and compared them with BIM of patients who did not progress to dysplasia/EAC (NDBE patients), both groups representing longitudinal cohorts (see FIG. 4 for representation of progression and longitudinal sampling of the patient groups).

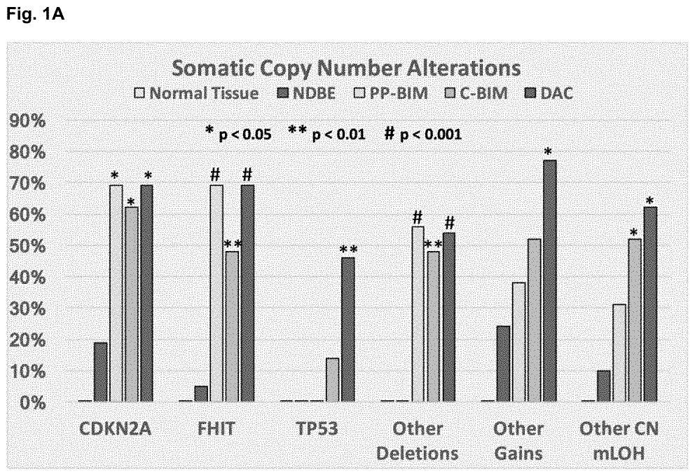

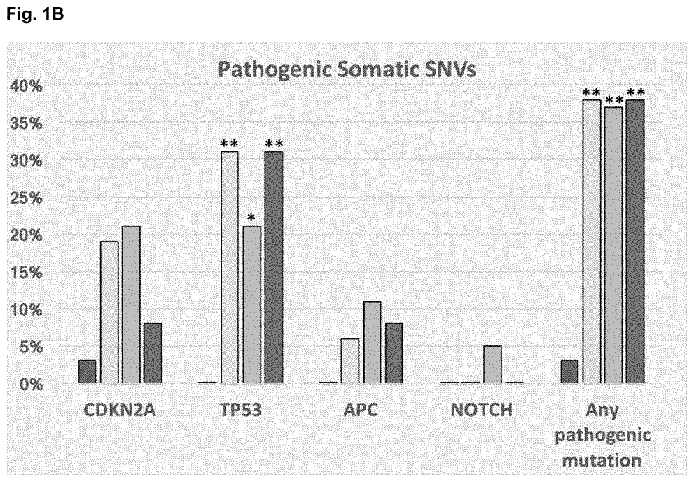

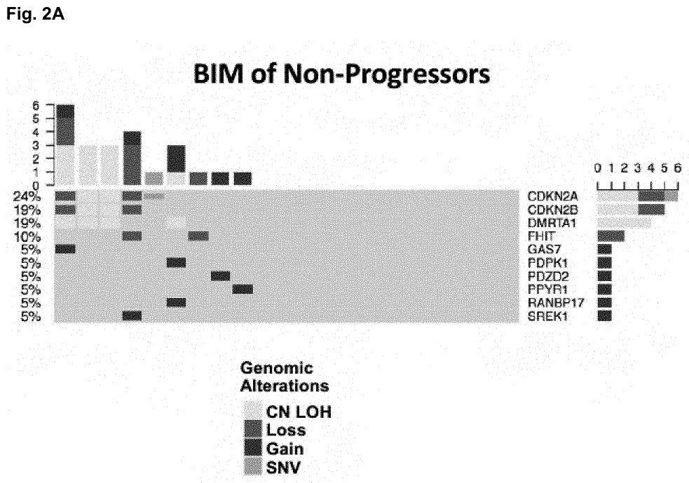

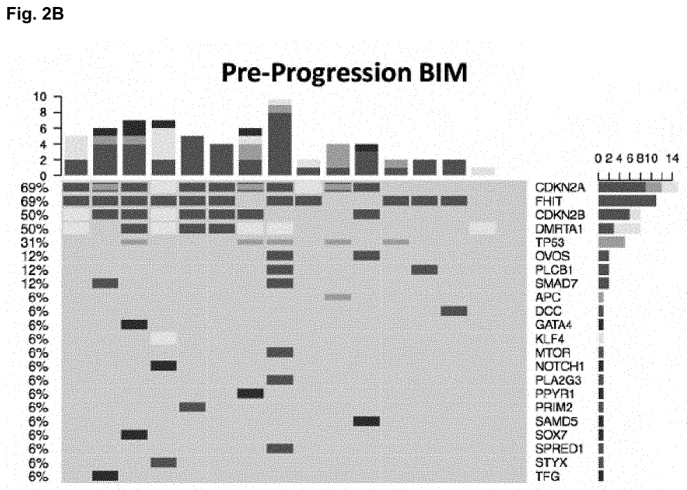

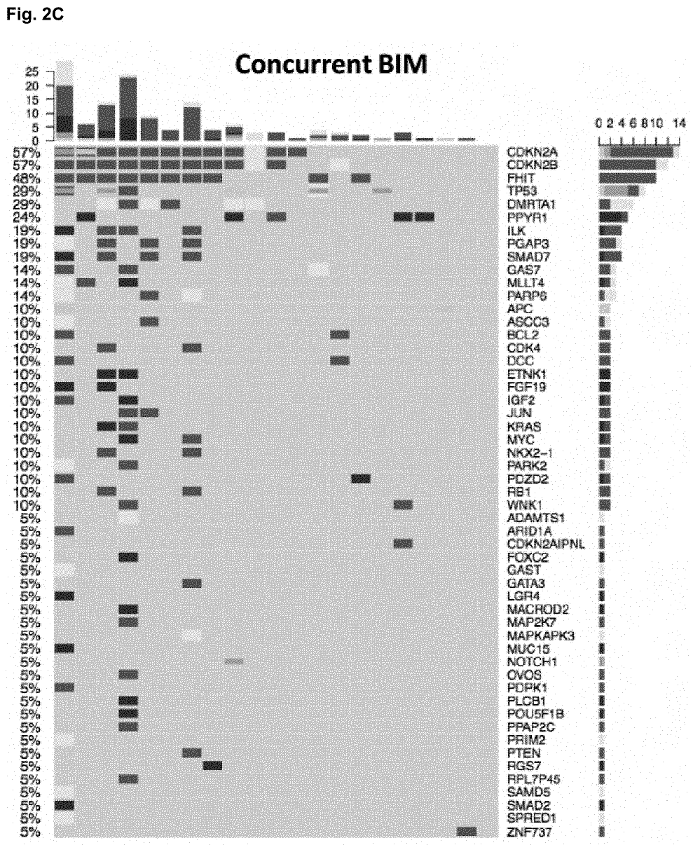

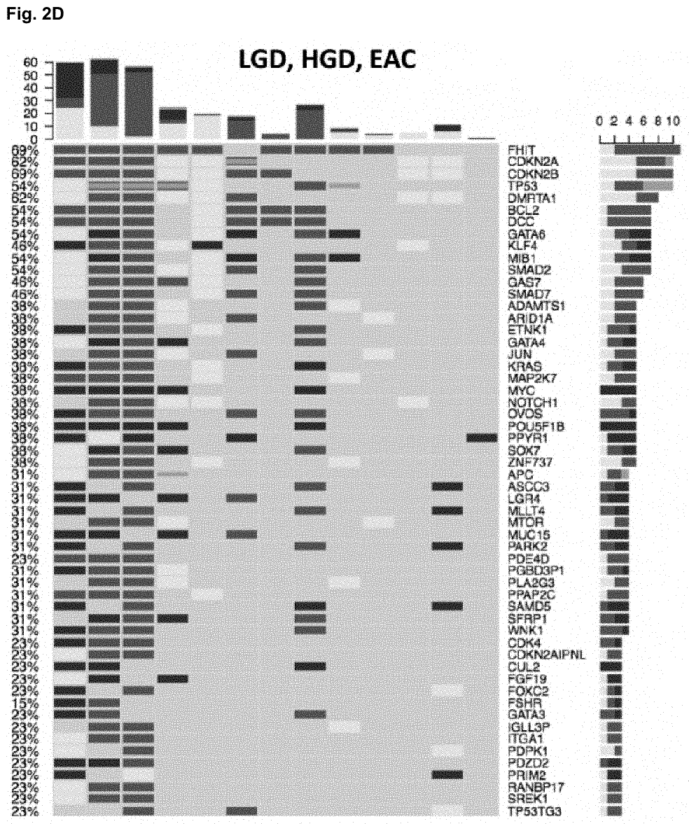

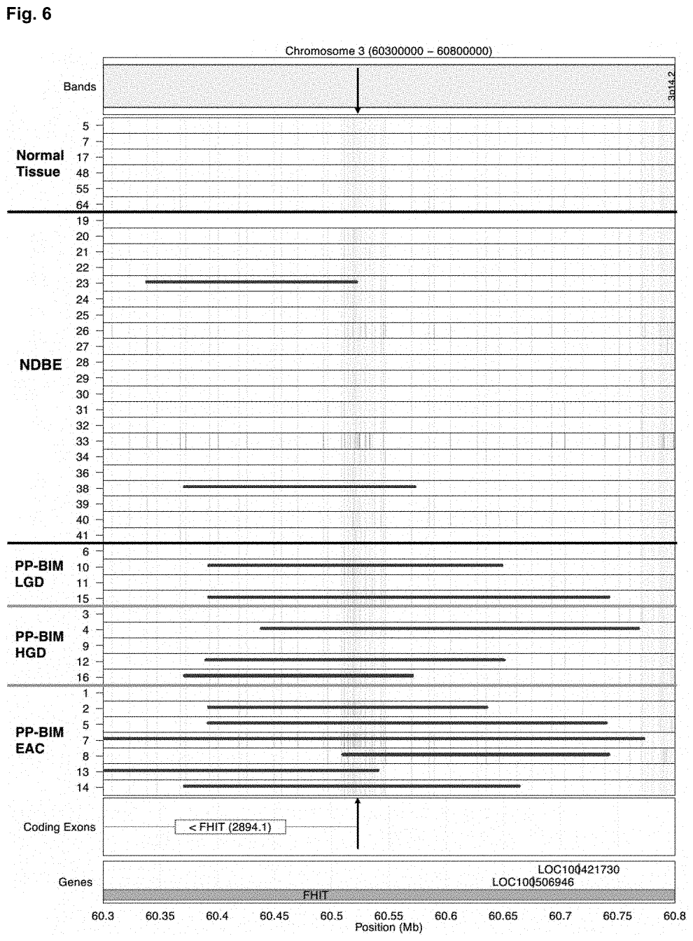

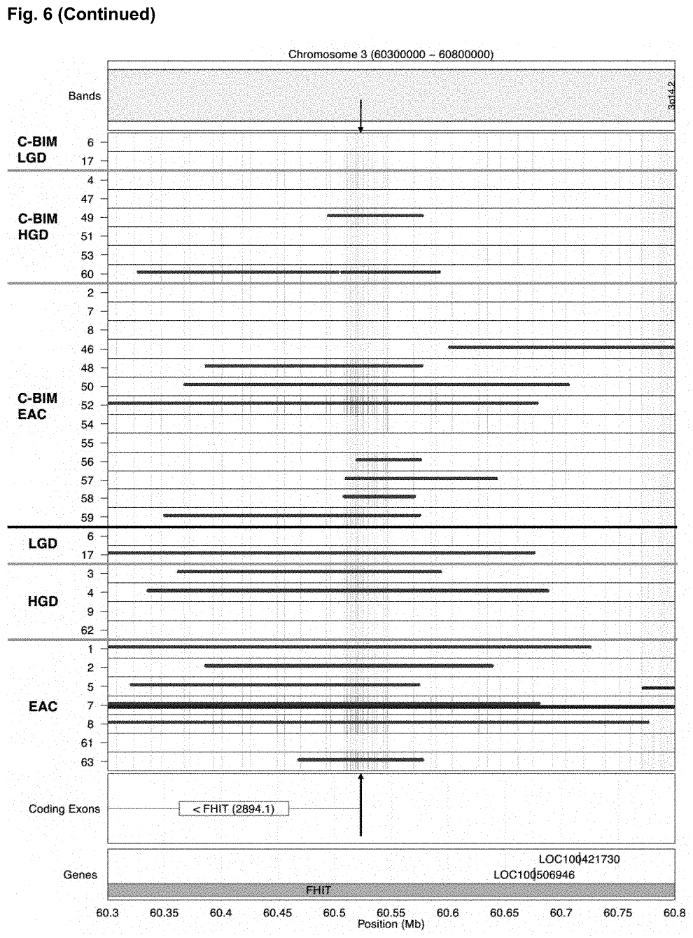

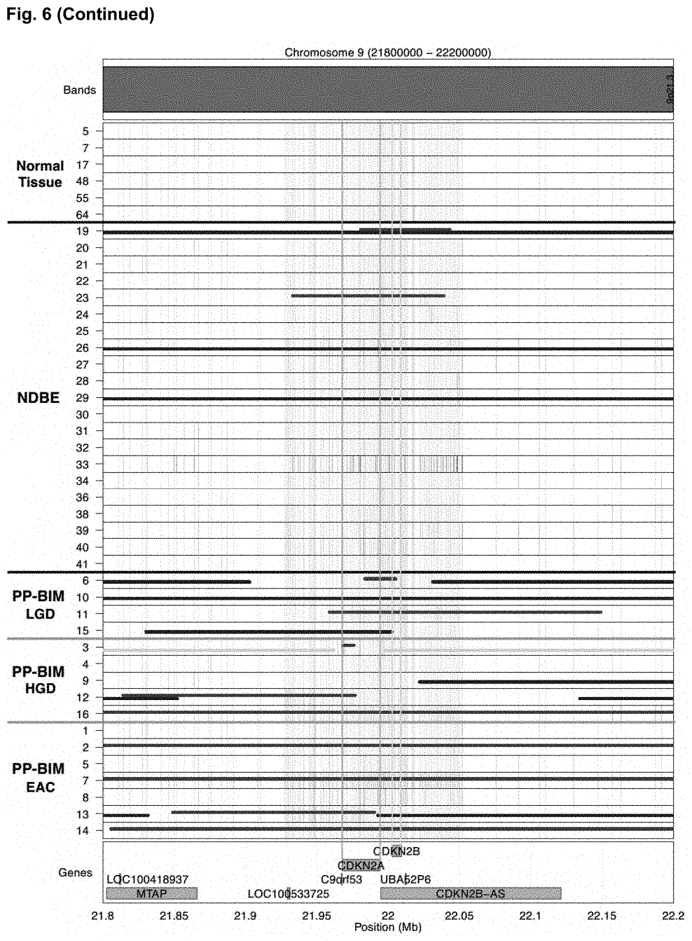

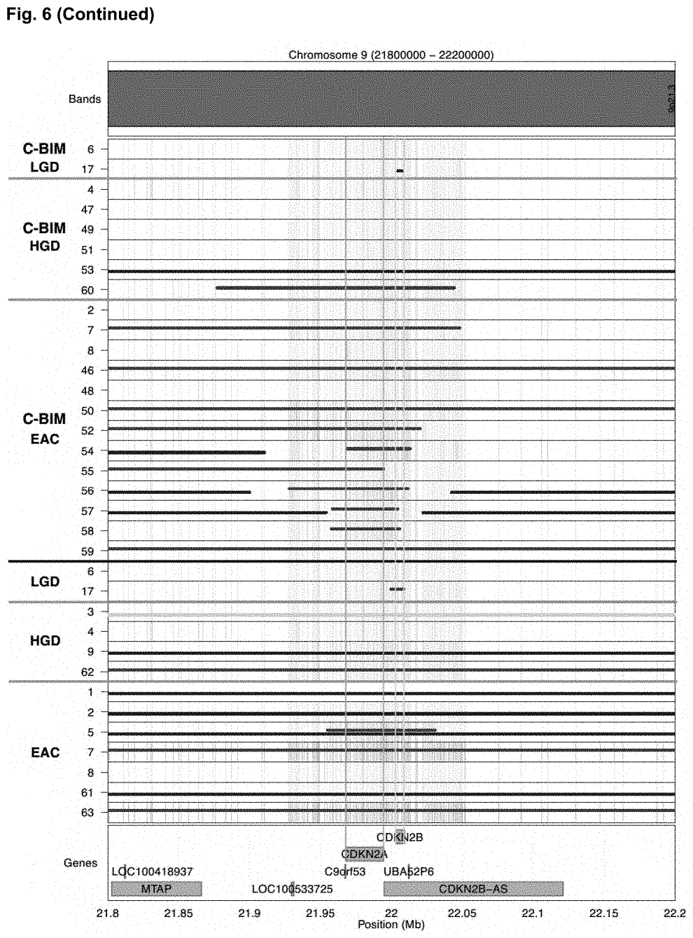

Thirty-seven patients had SNP-array results, 21 NDBE and 16 pre-progression-BIM (Tables 2 and 3, and FIGS. 1A, 2A-2D, and 5A-5C). Six patients with pre-progression-BIM also had non-esophageal normal tissues tested as controls, which showed no SCNAs. The most frequent SCNAs in pre-progression-BIM affected genomic regions that include the FHIT and CDKN2A genes. Deletion of FHIT, including exon 5, was found in 11/16 (69%) of pre-progression-BIM cases but only in 1/21 (5%) NDBE (p=0.0007) (see Table 3). The size of the deleted segments of FHIT involving exon 5 ranged from 195 to 614 kb (FIG. 6).

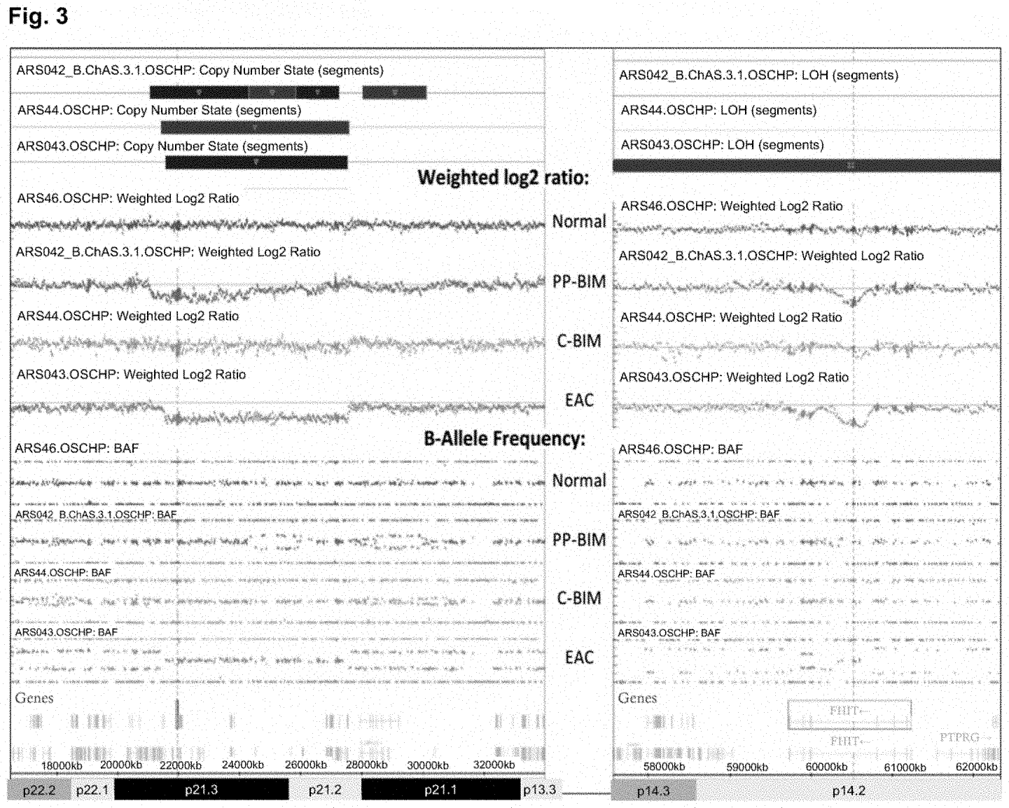

SCNAs in chromosome 9p including the CDKN2A gene were found in 11/16 cases (69%) of pre-progression-BIM and 4/21 (19%) NDBE patients (p=0.019). Hemizygous deletions of CDKN2A with partial (clonal) loss of heterozygosity (pLOH) were present in 25% of pre-progression-BIM but not in NDBE (p=0.02), whereas deletions without pLOH (likely homozygous) were present in 31% of pre-progression-BIM compared to 10% of NDBE. Copy neutral pLOH (representing clonal somatic uniparental disomy (Makishima et al 2011)) involving CDKN2A was detected in 13% of pre-progression-BIM and 10% of NDBE cases. The size of SCNAs in chromosome 9p ranged from a very focal deletion of 8 kb involving CDKN2A to the complete 9p arm (Table 2 and FIG. 6). FIGS. 3A and 3B show a representative progressor patient, comparing normal tissue showing no evidence of deletions with pre-progression-BIM carrying focal homozygous deletions of FHIT exon 5 and CDKN2A over three years before diagnosis of EAC. The EAC tissue showed the same FHIT deletion as well as extended homozygous deletion of CDKN2A, loss of one arm of chromosome 9p, and uniparental somatic duplication of the other arm of 9p.

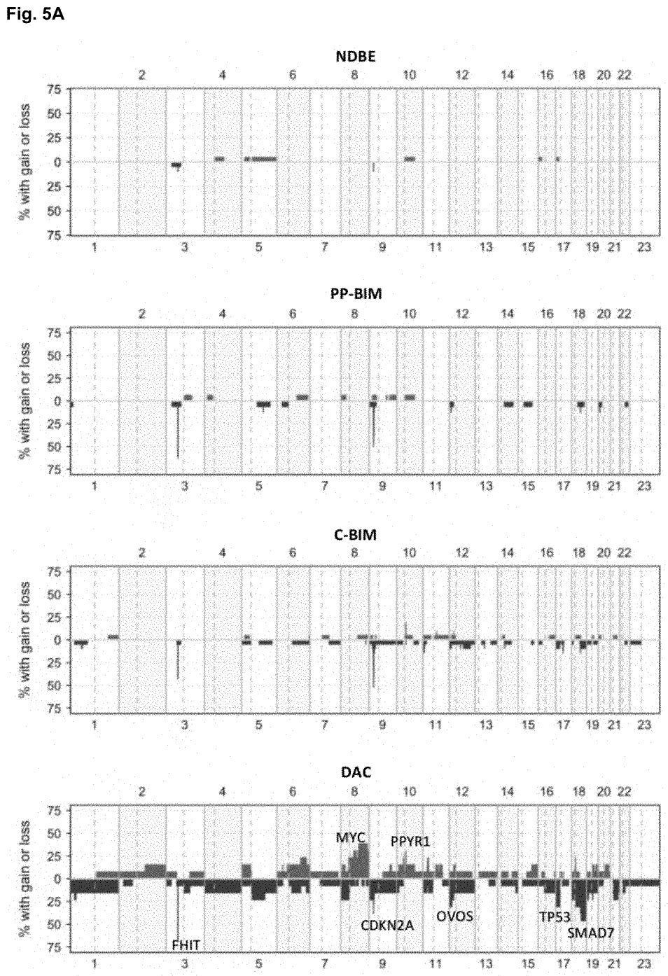

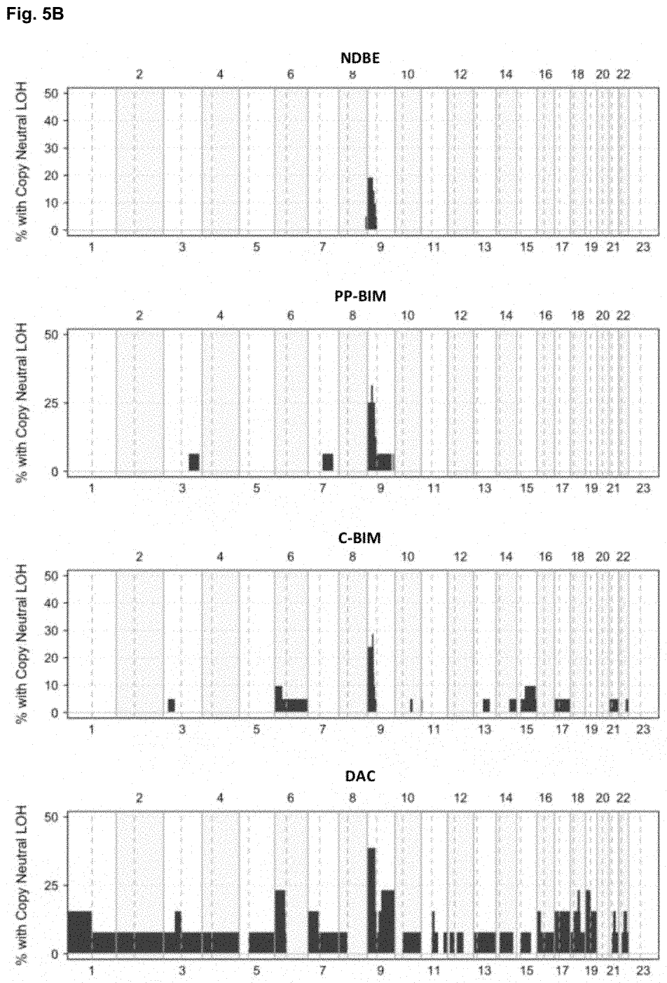

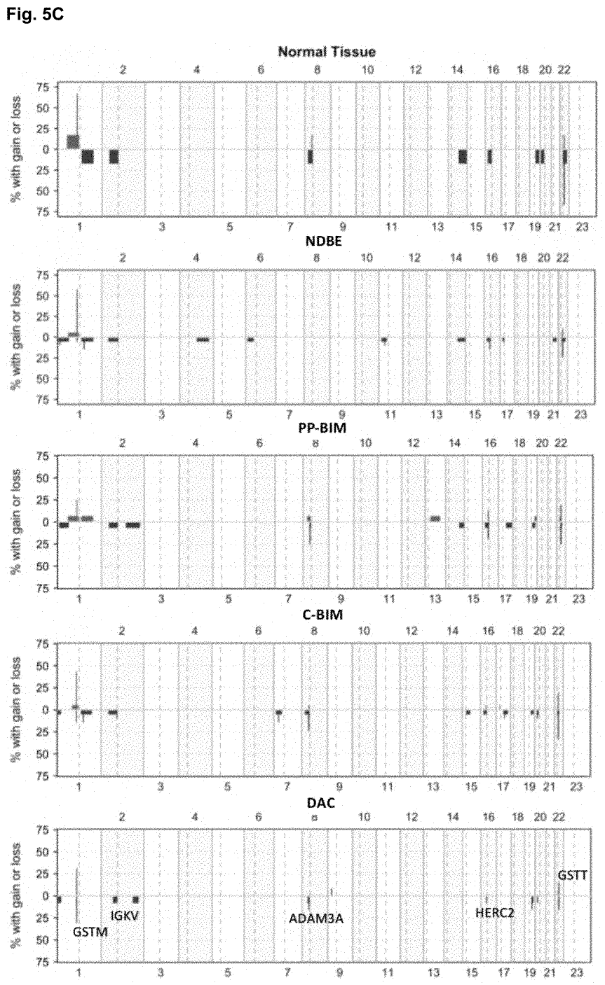

Other somatic deletions were found in 56% of pre-progression-BIM but not in NDBE (p=0.0006, Table 3), and included hemizygous deletions with pLOH in 6p11.2 and 9p, and deletions without pLOH in 5q22.2, 12p13.31, 14q22.1, 18q21.2, and 20p12.3 (Table 2 and FIGS. 5A to 5C). One pre-progression-BIM sample (patient 12) had somatic deletions in multiple chromosomes (Table 2). In addition, somatic gains were detected in 38% of pre-progression-BIM compared to 24% of NDBE, and copy neutral pLOH, frequently involving chromosome 9p, was seen in 44% of pre-progression-BIM and 19% of NDBE. No TP53 SCNAs were found in pre-progression-BIM or NDBE.

The average size of non-diploid genome, which was calculated as the sum of all somatic and germline deletions and gains divided by the size of the human genome, was significantly increased in pre-progression-BIM (0.11%) vs. NBDE (0.01%, P=0.03), but no differences were seen between NDBE and normal tissue (see Table 4).

The highest sensitivity for separating pre-progression-BIM of progressors from BIM of non-progressors was achieved with the combination of FHIT and CDKN2A SCNAs. FHIT and/or CDKN2A SCNAs were present in 88% of pre-progression-BIM compared to 24% of NDBE (p=0.007) (see Table 3). This results in a sensitivity of 88% (95% C.I=71-100%) and specificity of 76% (95% C.I.=58-94%) for separating pre-progression-BIM of progressors from BIM of non-progressors.

TABLE 2
Somatic copy number alterations (SCNA) in pre-progression-BIM of progressors, and in BIM of non-progressors.

| | | | | | |
|---|---|---|---|---|---|
| Patient | * FHIT | 9p Changes including | | | |
| No | Deletion | CDKN2A | Other Deletions | Gains | Copy neutral pLOH |

PP-BIM: Pre-Progression BIM (N = 16)

| | | | | | |
|---|---|---|---|---|---|
| 1 | ND | ND | ND | ND | ND |
| 2 | (262) | 9p24.3-21.3 Lp (20450) # | 6p11.2 Lp (PRIM2, 543) | 4p15.1 Gp (PCDH7, 1389); | ND |
| | | | | 9p22.3-13.2 Gp (16155) # | |
| 3 | ND | 9p21.3 Lc (8) | ND | ND | 3q26.1 (1529) |
| 4 | (356) | ND | 20p12.3 Ln (PLCB1, 177) | ND | ND |
| 5 | (375) | ND | 18q21.2 Lc (DCC, 287) | ND | ND |
| 6 | ND | 9p21.3 Lp (23) | ND | 10q11.22 Gp (PPYR1, 165) | 9p24.3-13.2 (36919) |
| 7 | (614) | 9p21.3 Ln (3197) | 9p21.3-21.1 Lp (3762) | 8p23.1 Gp (GATA4, 1283) | ND |
| 8 | (195) | ND | ND | ND | ND |
| 9 | ND | ND | ND | ND | 9p21.3 (DMRTA1, 3461) |
| 10 | (276) | 9p24.3-q34.12 CNm (109573) | 14q22.1 Ln (STYX, 214) | 9q21.33-34.3 Gn (31239) | 9p24.3-q34.12 (109573) |
| 11 | ND | 9p21.3 Lp (205) | 12p13.31 Ln (OVOS, 193) | 6q24.3 Gp (SAMD5, 319) | ND |
| 12 | (281) | 9p21.3 Lc (176) | \hat{ } | ND | 7q21.3 (1989); |
| | | | | | 9p24.3-13.1 (36541) |
| 13 | (531) | 9p21.3 Lc (153) | ND | ND | 9p24.3-13.3 (35476) |
| 14 | (316) | 9p21.3 Ln (493) | 18q21.1 8 Ln (SMAD7, 35) | 3q12.2 Gp (TFG, 137) | ND |
| 15 | (378) | 9p21.3 CNp (186) | 5q2.2 Lc (APC, 15) | ND | 9p21.3 (186) |
| 16 | (215) | 9p21.3 Lm (3647) | ND | ND | ND |
| TOTAL | 11 (69%) | 11 (69%) | 9 (56%) | 6 (38%) | 5 (32%) |
| B-BIMP (%) | | | | | |

NDBE: Never dysplastic BIM of Non-Progressors (N = 21)

| | | | | | |
|---|---|---|---|---|---|
| 19 | ND | 9p21.3 Ln (67) | ND | 4q13.1 Gn (LPHN3 198); | 9p24.3-13.3 (35929) |
| | | | | 17p13.1 Gp (GAS7 411) | |
| 22 | ND | ND | ND | 10q11.22 Gp (PPYR1 273) | ND |
| 23 | ND | 9p21.3 Lc (114) | ND | 5q12.3 Gp (SREK1 813) | ND |
| 26 | ND | 9p24.3-11.2 CNp (54571) | ND | ND | 9p24.3-11.2 (54571) |
| 28 | ND | ND | ND | 5p13.3 Gc (PDZD2 58) | ND |
| 29 | ND | 9p24.3-21.2 CNp (25609) | ND | ND | 9p24.3-21.2 (25609) |
| 30 | ND | ND | ND | 5q35.1 Gc (RANBP17 195), | 9p24.3-21.1 (19543) |
| | | | | 16p13.3 Gc (PDPK1 103) | |
| 38 | (217) | ND | ND | ND | ND |

20, 21, 24, 25, 27, 31-34, 36, 39-41: No SCNA Detected

| | | | | | |
|---|---|---|---|---|---|
| TOTAL | 1 (5%) | 4 (19%) | 0 (0%) | 5 (24%) | 2 (10%) |
| NDBE (%) | | | | | |

Note:
Parenthesis: representative genes found in smaller SCNA segments and size of SCNA segments in Kb.
* Deletion of 3p14.2 including FHIT exon 5. ND: not detected; Ln: Copy number loss without loss-of-heterozygosity (LOH); Lp: Loss with partial (clonal) LOH; Lc: Loss in segment of complete LOH; Gn: Gain without LOH; Gp: Gain with partial LOH; Gc: Gain in segment of complete LOH; CNp: Copy neutral partial LOH.
\hat{ } - Multiple deletions: 1p36.22 Lc (MTOR, 12); 5q22.2 Ln (237); 9p13.3 Lp (1232); 12p13.31 Lc (OVOS, 248); 15q14 Lc (SPRED1, 61); 18q21.1 Lc (SMAD7, 56); 20p12.3 Ln (PLCB1, 178); 22q12.2 Lc (PLA2G3, 202).
# Complex interspersed deletions and gains in 9p genomic region.

TABLE 3
Summary data of SNP arrays for each group of samples.

| | Normal Tissue | NDBE | PP-BIM | C-BIM | DAC |
|---|---|---|---|---|---|
| | (N = 6) | (N = 21) | (N = 16) | (N = 21) | (N = 13) |
| FHIT Deletion Exon 5 | 0 (0%) | 1 (5%) | 11 (69%) | 10 (48%) | 9 (69%) |
| | | | [0.0007] | [0.0067] | [0.0008] |
| CDKN2A/2B | 0 (0%) | 4 (19%) | 11 (69%) | 13 (62%) | 9 (69%) |
| | | | [0.0187] | [0.0290] | [0.0215] |
| Homozygous deletion or deletion in area | 0 (0%) | 2 (10%) | 5 (31%) | 5 (24%) | 2 (15%) |
| of complete LOH | | | [0.1323] | [0.2568] | [0.6283] |
| Hemizygous deletion | 0 (0%) | 0 (0%) | 4 (25%) | 6 (29%) | 3 (23%) |
| | | | [0.0219] | [0.0143] | [0.0277] |
| Copy neutral partial LOH | 0 (0%) | 2 (10%) | 2 (13%) | 2 (10%) | 4 (31%) |
| | | | [0.7850] | [1.0000] | [0.1518] |
| TP53 | 0 (0%) | 0 (0%) | 0 (0%) | 3 (14%) | 6 (46%) |
| | | | | [0.0833] | [0.0019] |
| Other Somatic Deletions | 0 (0%) | 0 (0%) | 9 (56%) | 10 (48%) | 7 (54%) |
| | | | [0.0006] | [0.0016] | [0.0008] |
| Somatic Gains | 0 (0%) | 5 (24%) | 6 (38%) | 11 (52%) | 10 (77%) |
| | | | [0.4493] | [0.1336] | [0.0235] |
| Somatic Copy Neutral mLOH | 0 (0%) | 4 (19%) | 7 (44%) | 12 (57%) | 9 (69%) |
| | | | [0.1722] | [0.0455] | [0.0215] |
| Any FHIT or CDKN2A SCNA | 0 (0%) | 5 (24%) | 14 (88%) | 14 (67%) | 12 (92%) |
| | | | [0.0074] | [0.0389] | [0.0061] |
| Any FHIT or CDKN2A SCNA or Other | 0 (0%) | 5 (24%) | 14 (88%) | 17 (81%) | 12 (92%) |
| Somatic Deletions | | | [0.0074] | [0.0105] | [0.0061] |

Note:
NDBE: BIM from never dysplastic Barrett's esophagus; PP-BIM: pre-progression-BIM from patients who subsequently (greater than one year later) progressed to dysplasia or EAC; C-BIM: BIM from temporally concurrent but spatially separate specimens of patients with dysplasia or EAC; DAC: samples from dysplasia or EAC. In each of the groups listed in each column, there was one sample tested per patient. Individual cells in the table contain the number of samples with each type of somatic copy number alteration (SCNA) listed on the left, the percent of positive samples in each group (in round parenthesis), and the Chi-square p-value in comparison to NDBE (in square parenthesis).
Statistically significant SCNA (p < 0.05) are highlighted in bold.

TABLE 4
Average percent of non-diploid genome (±standard
error of the mean) in each group of samples.

| | Average % Non- | P vs. | P vs. | P vs. |
|---|---|---|---|---|
| GROUP | diploid genome | NDBE | PP-BIM | C-BIM |
| Normal Tissue | 0.01% ± 0.01% | | | |
| NDBE | 0.01% ± 0.01% | | | |
| PP-BIM | 0.11% ± 0.20% | 0.032 | | |
| C-BIM | 2.01% ± 6.64% | 0.176 | 0.262 | |
| Dysplasia or EAC | 6.09% ± 9.93% | 0.006 | 0.023 | 0.159 |

Note:
Statistical significant results (2-sided t-test) are highlighted in bold. Abbreviations as in Table 1.

### Example 3

### Somatic Genomic Copy Number Alterations in Non-Dysplastic Concurrent Intestinal Metaplasia, Dysplasia, and Adenocarcinoma Lesions of Progressors

Twenty-one cases of concurrent-BIM were tested with SNP arrays (Table 1). Six patients were from the progressor group with baseline pre-progression-BIM tested. Fifteen additional concurrent-BIM lesions tested were from patients without baseline pre-progression-BIM available. Thirteen dysplasia/EAC lesions were tested with SNP arrays; ten were from progressor patients with baseline pre-progression-BIM and 3 did not have pre-progression-BIM tissues available (Table 1). The tested EAC lesions were all intramucosal adenocarcinomas, except cases 8 (pT1b) and 5 (pT3).

As summarized in Table 3 and detailed in Tables 5 and 6, losses of 3p14.2 involving FHIT exon 5 were present in only one NDBE patient (5%), and were more frequent in dysplasia/EAC (69%, P=0.0008 NDBE) and concurrent-BIM (48%, P=0.007 vs. NDBE). Likewise, deletions or copy neutral pLOH of chromosome 9p involving CDKN2A/B were present in 19% of NDBE but were more frequent in dysplasia/EAC (69%, P=0.02 vs. NDBE) and concurrent-BIM (62%, P=0.03 vs. NDBE). Copy number gains were more frequently seen in dysplasia/EAC (77%) and concurrent-BIM (52%) than NDBE (38%) (FIGS. 1A, 2A to 2D, and 5A to 5C).

In addition to the alterations in 3p14.2 involving FHIT exon 5 and in 9p involving CDKN2A/B, somatic gene losses were detected in 18q21.1-18q21.2 including BCL2 and SMAD7, 17p13.1 (TP53), 11p15.4(ILK) and 19p13.2(MAP2K7) and gains in 8q24.21(MYC) and 6q24.2-6q25.1(SAMD5) in dysplasia/EAC and concurrent-BIM but not in NDBE.

SCNA alterations in dysplasia/EAC lesions (Table 6) were more extensive than in concurrent-BIM (Table 5). The average fraction of non-diploid genome was low in NDBE (0.01%), increased in concurrent-BIM (2.0%), and was highest in dysplasia/EAC (6.1%, p=0.008 vs. NDBE). The average fraction of non-diploid genome was higher in EAC (10%) than in low and high-grade dysplasias (1.8%).

TABLE 5
SCNA alterations in concurrent-BIM.

| Patient | | | |
|---|---|---|---|
| N = 21 | FHIT * | 9p Changes Including CDKN2A/B | Other Deletions |
| 2 C | ND | ND | ND |
| 4 H | ND | ND | ND |
| 6 L | ND | ND | ND |
| 7 C | ND | 9p21.3 Ln (CDKN2A/B, 352) | 10q11.22 Lc (PPYR1, 154) |
| 8 C | ND | ND | ND |
| 17 L | ND | 9p21.3 CNp (CDKN2B, 2520) | 18q21.2-23 Lp (27542) |
| 46 C | (417) | 9p22.2-21.3 Lp (CDKN2A/B, 2454) | 6q25.2-27 Lp (6300) |
| 47 H | ND | ND | 19p12 Lc (ZNF737, 130) |
| 48 C | (205) | ND | ND |
| 49 H | (89) | ND | ND |
| 50 C | (365) | 9p21.3-21.2 Lp (CDKN2A/B, 5362) | ND |
| 51 H | ND | ND | 5q31.1 Ln (CDKN2AIPNL, 86); |
| | | | 12p13.33 Ln (WNK1, 15) |
| 52 C | (447) | 9p21.3 Lp (CDKN2A/B, 767) | ND |
| 53 H | ND | 9p24.3-21.1 CNp (CDKN2A/B, 28238) | ND |
| 54 C | ND | 9p21.3 Ln (CDKN2A/B, 48) | ND |
| 55 C | ND | 9p21.3 Lc (CDKN2A, 306) | ND |
| 56 C | (60) | 9p21.3 Lp (CDKN2A/B, 90) | 1p32.1 Lc (JUN, 12); |
| | | | 6q16.3 Ln (ASCC3, 87); |
| | | | 15q23 Lc (PARP6, 102); |
| | | | 17q12 Lc (PGAP3, 174); |
| | | | 18q21.1 Lc (SMAD7, 230) |
| 57 C | (143) | 9p21.3 Lc (CDKN2A/B, 49) | 11p15.4 Ln (ILK, 88); |
| | | | 13q14.2 Lc (RB1, 44); |
| | | | 17q12 Lc (PGAP3, 176) |
| 58 C | (67) | 9p21.3-33.3 Lc (CDKN2A/B, 337) | 8q24.21 Lc (MYC, 23); |
| | | | 10p14, p12.31, q23.31 Lc |
| | | | (GATA3, MLLT10, PTEN, 139); |
| | | | 12q14.1-14.3 Lc (CDK4, 43); |
| | | | 13q14.2 Lc (RB1, 46); |
| | | | 14q13.3 Ln (NKX2-1, 86); |
| | | | 17q12 Lc (PGAP3, 174); |
| | | | 18q21.1 Lc (SMAD7, 10); |
| | | | 22q12.1-12.2 Lc (202) |
| 59 C | (243) | 9p24.3-q31.1 Lp (CDKN2A/B, 103069) | 1p32.3-31.3 Lp (9825); |
| | | | 6q26 Lp (PARK2, 476); |
| | | | 7q22.1-36.3 Lp (60357); |
| | | | 11p15.4 Lp (6156); |
| | | | 12p13.33-q24.33 Lp (111318); |
| | | | 13q33.1-33.2 Lp (RPL7P45, 1798); |
| | | | 17p13.3-12 Lp (TP53, 15048); |
| | | | 18p11.33 Lp (469); |
| | | | 19p13.3-q13.43 Lp (31236) |
| 60 H | (428) | 9p21.3-21.1 Lp (CDKN2A/B, 6939) | 1p36.11 Lp (ARID1A, 349); |
| | | | 5p15.33-p11 Ln (46347); |
| | | | 11p15.5-15.4 Lp (IGF2, 2593); |
| | | | 12q21.2-21.31 Lp (9627); |
| | | | 16p13.3 (6450); |
| | | | 17p13.3-11.1 Lp (TP53, 22866); |
| | | | 18q21.1-23 Lp (31044); |
| | | | Xp22.33-11.21 Lp (56247) |
| TOTAL (%) | 10 (48%) | 12 (57%) | 10 (48%) |

| Patient | | |
|---|---|---|
| N = 21 | Gains | Copy neutral partial LOH |
| 2 C | 9p22.3-22.2 Gp (1429) | 9p13.3-13.2 (1616) |
| | 9p21.1-13.2 Gp (5504) | |
| 4 H | ND | ND |
| 6 L | 10q11.22 Gp (PPYR1, 167) | ND |
| 7 C | ND | 9p24.3-13.1 (33329) |
| 8 C | ND | ND |
| 17 L | ND | 14q32.33 (968); |
| | | 9p21.3 (2520) |
| 46 C | 10q11.22 Gn (PPYR1, 1302) | ND |
| 47 H | | ND |
| 48 C | ND | 17p13.3-11.2 (TP53, 20251) |
| 49 H | 5p13.3 Gn (PDZD2, 56) | ND |
| 50 C | ND | ND |
| 51 H | 10q11.22 Gp (PPYR1, 624) | ND |
| 52 C | 1q43 Gc (RGS7, 102) | 9p21.3-21.1 (5176) |
| 53 H | ND | 9p24.3-21.1 (28238) |
| 54 C | 10q11.22 Gp (PPYR1, 614) | 9p24.3-21.1 (32845) |
| 55 C | ND | ND |
| 56 C | ND | 9p24.3-21.1 (32957) |
| 57 C | 11q13.3 Gc (FGF19, 47); | 9p24.3-21.1 (30102) |
| | 11q13.4 Gp (1605); | |
| | 12p12.1 Gp (KRAS, 4433) | |
| 58 C | ND | 3p21.31-21.2 (MAPKAPK3, 728); |
| | | 15q22.2-25.3 (23455) |
| 59 C | 6q27 Gp (MLLT4, 272) | 6p25.3-22.3 (17536); |
| | 7q11.23-22.1 Gp (25734); | 10q23.1-23.2 (6663); |
| | 8q24.21 Gp (POU5F1B, 569); | 11p15.5-15.4 (2827); |
| | 10q22.1-22.3 Gp (5025); | 21p11.2-q22.3 (38566); |
| | 11p15.5 Gn (IGF2, 137); | 22q13.31-13.33 (3356) |
| | 12p12.1 Gp (ETNK1, 514); | |
| | 16q23.2-24.1 Gp (FOXF1/C2/L1, 3962); | |
| | 19q11-13.12 Gp (8028); | |
| | 20p13-12.1 Gp (14632); | |
| | 21q11.2-21.1 Gp (5356) | |
| 60 H | 11p15.4-q25 Gp (45707); | 6p25.3-q27 (170260); |
| | 14q13.1-13.2 Gp (1197); | 13q14.3-21.1 (6644); |
| | 18q12.1-21.1 Gp (SMAD7, 17755); | 15q11.2-26.3 (80678); |
| | 21q11.2-21.1 Gp (5356) | 17q11.1-25.3 (55927) |
| TOTAL (%) | 11 (52%) | 12 (57%) |

Note:
SNP array results from temporally concurrent-BIM (C-BIM) areas of non-dysplastic intestinal metaplasia spatially separate from co-existing low-grade dysplasia (denoted by L next to patient number), high-grade dysplasia (denoted by H next to patient number), or esophageal adenocarcinoma (denoted by C next to patient number). Values in parenthesis are representative genes in smaller SCNA and the size of the SCNA in Kb. ND: not detected; Ln: Loss without LOH; Lp: Loss with partial (clonal) LOH; Lc: Loss in complete LOH; Gn: Gain without LOH; Gp: Gain with partial LOH; Gc: Gain in complete LOH; CNp: Copy neutral partial LOH.
* 3p14.2 Deletion Including FHIT Exon 5.

TABLE 6
SCNA alterations in dysplasia/EAC lesions.

| Patient | | | |
|---|---|---|---|
| N = 13 | FHIT * | 9p Changes Including CDKN2A/B | Other Deletions |
| 1 C | (668) | 9p24.3-9q34.3 CNp (CDKN2A/B, 109622) | ND |
| 2 C | (273) | 9p24.3-q34.3 CNp (CDKN2A/B, 38772) | 17p13.3-11.2 Lp (TP53, 21832) |
| 3 H | (250) | ND | ND |
| 4 H | (381) | ND | ND |
| 5 C | (273) | 9p21.3 Ln (CDKN2A/B, 82) | 3q11.1-13.31 Lp (17984); |
| | | | 5q11.2 Ln (PDE4D, 256); |
| | | | 16p11.1-q24.1 Lp (33406); |
| | | | 18q21.2-23 Lp (28030); |
| | | | 19p13.2-q12 Lp (5370); |
| | | | 19p13.3 Ln (PPAP2C, 64) |
| 6 L | ND | ND | ND |
| 7 C | (363) | 9p24.3-q34.3 Lp (CDKN2A/B, 134738) | 1p36.33-q44 Lp (247940); |
| | | | 2p16.3 Lp (FSHR, 225); |
| | | | 3p26.3-q25.33 Lp (8004); |
| | | | 4p16.3-q21.21 Lp (80961); |
| | | | 4q12-35.2 Ln (116589); |
| | | | 5q11.2 Lp (ITGA1/A2, 1365); |
| | | | 5q11.2-35.3 Ln (127780); |
| | | | 6p25.3-24.1 Lp (11539); |
| | | | 6p24.1-21.33 Ln (19105); |
| | | | 7p11.2-q36.3 Lp (101991); |
| | | | 8p23.3-q24.3 Lp (82903); |
| | | | 9p21.3-21.2 Ln (6024); |
| | | | 10p15.3-11.22 Ln (34386); |
| | | | 11p15.5-q25 Lp (75192); |
| | | | 12p13.33-q24.33 Lp (135007); |
| | | | 14q11.2-14q32.33 Lp (80010); |
| | | | 17p13.3-11.1 Lp (TP53, 21853); |
| | | | 18p11.32-q23 Lp (71158); |
| | | | 19p13.3-11 Lp (24493); |
| | | | 19q11-13.43 Ln (31368); |
| | | | 21q11.2-22.3 Lp (33582); |
| | | | 22q11.1-13.31 Lp (31327); |
| | | | Xp22.33-q28 Lp (155271) |
| 8 C | (512) | ND | 1p36.11-35.3 Lp (1954); |
| | | | 6q14.3-q27 Lp (51805); |
| | | | 8p23.3-11.1 Lp (44154); |
| | | | 10p15.3-12.31 Lp (22556); |
| | | | 12p13.33-12.1 Lp (23866); |
| | | | 13q31.1-31.3 Lp (11208); |
| | | | 17p13.3-11.2 Lp (TP53, 21682); |
| | | | 18p11.32-q22.2 Lp (68226); |
| | | | 20p13-11.1 Lp (25409); |
| | | | 20p12.1 Ln (MACROD2, 154); |
| | | | 21q11.2-22.11 Lp (16260) |
| 9 H | ND | 9p24.6-q34.3 CNp (CDKN2A/B, 141218) | ND |
| 17 L | (611) | 9p21.3 Lp (CDKN2A/B, 28061) | 14q32.2-32.33 Lp (6696); |
| | | | 18q21.2-23 Lp (27192) |
| 61 C | ND | 9p24.3-13.1 CNp (CDKN2A/B, 39329); | ND |
| 62 H | ND | 9p24.3-21.3 Lp (CDKN2A/B 24532) | 11p15.5-11.12 Lp (32026); |
| | | | 12p13.31 Ln (OVOS, 163); |
| | | | 16p11.2 Lc (TP53TG3/3B, 1049); |
| | | | 18q11.2-q23 Lp (57813) |
| 63 C | (118) | 9p21.3 Ln (CDKN2A/B, 2302); | 1p36.33-q44 Lp (248908); |
| | | | 3p14.2-q29 Lp (94935); |
| | | | 4p16.3-q35.2 Lp (193400); |
| | | | 5p15.33-q35.3 Lp (180690); |
| | | | 6p25.3-q12 Lp (107121); |
| | | | 8p23.3-11.21 Lp (41237); |
| | | | 9p24.3-q34.3 Lp (137976); |
| | | | 12p13.33-q24.33 Ln (135007); |
| | | | 15q11.2-26.3 Lp (79470); |
| | | | 16p13.3-q24.3 Lp (903298); |
| | | | 17p13.3-q21.32 Lp (TP53, 30904); |
| | | | 18p11.32-11.1 Ln (15560); |
| | | | 18q11.1-23 Lp (59264); |
| | | | 19p13.3-q13.43 Lp (55943); |
| | | | 21q11.2-22.3 Lp (33739); |
| | | | 22q11.23-12.1 Ln (IGLL3P, 255); |
| | | | 22q12.1-11.23 Lp (35070) |
| TOTAL (%) | 9 (69%) | 9 (69%) | 7 (54%) |

| Patient | | |
|---|---|---|
| N = 13 | Gains | Copy neutral mLOH |
| 1 C | 9q31.2-31.3 Gp (KLF4, 1885); | 6p25.3-22.2 (26005); |
| | 19p13.3-q13.43 Gp (11613); | 7p22.3-q36.3 (171115); |
| | 20q13.12 Gp (3350) | 12p12.3-11.1 (10967); |
| | | 17p13.3-11.1 (TP53, 22397); |
| | | 18p11.32-q23 (81185); |
| | | 19p13.3-q13.43 (78077) |
| 2 C | 8p23.3-q24.3 Gp (144704); | 1p36.33-q44 (248939); |
| | 9q13-23.3 Gn (34025) \hat{ } | 9q21.13-34.3 (38053) \hat{ } |
| | 11p14.3-q13.4 Gp (5532) | 11q25 (3822); |
| | | 12q14.1 (PGBD3P1, 1140); |
| | | 14q24.3 (1386); |
| | | 18q12.3-21.1 (SAMD2, 2094); |
| | | 21q21.3 (ADAMTS1/5, 2318); |
| | | 22q12.2-12.3 (2947) |
| 3 H | ND | 1p36.33-11.2 (120655) |
| 4 H | 18q11.2-12.2 Gp (16104) | 19p13.3-q13.43 (55948); |
| | | 21q11.2-22.3 (30300); |
| | | 22q11.21-13.33 (31641) |
| 5 C | 1q21.1-43 Gp (95219); | 3p14.2-11.1 (29078); |
| | 2p25.3-q37.3 Gp (249251); | 4p16.3-q35.2 (191154); |
| | 3p26.3-q29 Gp (115856); | 5q11.1-35.3 (137688); |
| | 5p13.2-12 Gn (9421); | 6p25.3-12.1 (54190); |
| | 5p15.33-13.2 Gp (36177); | 8p23.3-11.21 (40914); |
| | 6p12.1-q12 Gn (12070); | 9p24.3-13.1 (38597); |
| | 6q12-27 Gp (103750); | 11q12.1-14.1 (25804); |
| | 7p22.3-11.1 Gn (58027); | 14q11.2-24.3 (54936); |
| | 7q11.1-32.3 Gp (69678); | 15q11.1-22.31 (44722); |
| | 8p11.21-q24.3 Gp (105084); | 16p13.3-12.3 (18324); |
| | 9q21.11-34.11 Gp (61359); | 17p13.3-11.1 (TP53, 22209); |
| | 10p15.3-q11.21 Gn (42909); | 18q11.1-18q21.2 (31492); |
| | 10q11.22-26.3 Gp (88896); | 19p13.3-19p11 (21800) |
| | 11p15.4-q12.1 Gn (50652); | |
| | 11q14.1-21 Gp (12317); | |
| | 12p13.33-q23.1 Gp (92894); | |
| | 12q23.1 Gn (3680); | |
| | 13q11-34 Gp (96111); | |
| | 14q24.3-31.3 Gp (12352); | |
| | 14q31.3-q32.33 Gn (19338); | |
| | 15q22.31-26.3 Gn (50388); | |
| | 16p12.3-11.2 Gn (13626) | |
| | 16q24.1-24.3 Gp (6062); | |
| | 17q11.1-11.2 Gp (TP53, 3721); | |
| | 19q12-13.43 Gp (27525); | |
| | 20p13-q13.33 Gp (59691); | |
| 6 L | 10q11.22 Gp (PPYR1, 182) | ND |
| 7 C | 6p21.33-21.32 Gc (997); | 2p25.3- q37.3 (241936); |
| | 5p15.33-q11.2 Gp (52553); | 3p26.1-q29 (193155); |
| | 8p23.1-8q24.23 Gp (53453); | 6p21.32-12.3 (17109); |
| | 8p11.21 Gn (SFRP1, 2244); | 7p22.3- 12.3 (48719); |
| | 10p11.2; 10p11.21-10p11.1 Gp (4668); | 10q11.21-26.3 (93018); |
| | 11p14.3-11q22.1; 11q22.1 Gp (44023); | 11q12.1-13.2 (8999); |
| | 18q11.1-18q11.2 Gp (GATA6, 3806) | 13p13-q34 (133852); |
| | | 14q31.1-31.3 (5090); |
| | | 17q11.1-25.3 (55136) |
| 8 C | 2q31.1-37.3 Gp (64061); | ND |
| | 6q22.31-25.2 Gp (25316); | |
| | 8q21.3-8q24.13 Gp (34085); | |
| | 8q24.13-8q24.3 Gn (21000); | |
| | 10p12.31-10p11.21 Gp (13946); | |
| | 12p12.1; 12p12.1-2p11.22 Gp (5103); | |
| | 14q11.2; 14q12-14q13.1 Gp (2717) | |
| 9 H | ND | 9p24.3-p34.3 (141218) |
| 17 L | ND | ND |
| 61 C | 6p25.3-q27 Gp (171115) | 16p13.3-q24.3 (90355) |
| 62 H | 10p11.22 Gn (PPYR1, 746); | ND |
| | 15q24.1-15q26.3 Gp (27454); | |
| | 18p11.32-18q11.2 Gp (17109) | |
| 63 C | 8q11.1-24.3 Gp (99596); | 6p25.3-11.2 (57258); |
| | 10q11.22 Gp (PPYR1, 738); | 17q11.2-25.3 (43630) |
| | 17p11.2-q21.2 Gp (GAST, 1007) | |
| TOTAL (%) | 10 (77%) | 9 (69%) |

Note:
SNP arrays from areas of dysplasia (low-grade, L, and high-grade, H, next to patient's number) or EAC (C next to patient's number). Values in parenthesis are representative genes in smaller SCNA and the size of the SCNA in Kb. ND: not detected; Ln: Loss without LOH; Lp: Loss with mosaic LOH; Lc: Loss in complete LOH; Gn: Gain without LOH; Gp: Gain with mosaic LOH; Gc: Gain in complete LOH; CNp: Copy neutral mosaic LOH.
* 3p14.2 Deletion Including FHIT Exon 5.
\hat{ } Segments with LOH and interspersed areas of amplification.
# Segments with LOH and interspersed areas of deletion.

### Example 4

### Somatic Mutations and SCNAs in Pre-Progression Non-Dysplastic Barrett's Intestinal Metaplasia, Non-Dysplastic Concurrent Intestinal Metaplasia, Dysplasia and EAC Lesions

Sixty-five samples were tested by NGS targeted panels, including 27 non-progressors (NDBE), 16 pre-progression-BIM of progressors, 19 concurrent-BIM of progressors, and 13 dysplasia/EAC lesions (see Table 7, Table 8, and FIG. 1B). Mutations detected by NGS were present in 6 (38%) pre-progression-BIM (3 in CDKN2A, 5 in TP53, and 1 in APC) as compared to 3% of NDBE (1 CDKN2A mutation), p=0.008. There were no significant differences in the frequencies of mutations detected by NGS in pre-progression-BIM (38%), concurrent-BIM (37%), and dysplasia/EAC (38%). Compared to SNP-arrays alone, the combination of NGS and SNP arrays did not detect additional samples with genomic alterations in pre-progression-BIM or dysplasia/EAC. Combining NGS and SNP array results slightly increased the detection of genomic changes in concurrent-BIM (86% combined vs. 81% SNP arrays alone), without increasing the number of NDBE cases with genomic alterations (24%) (see Table 7).

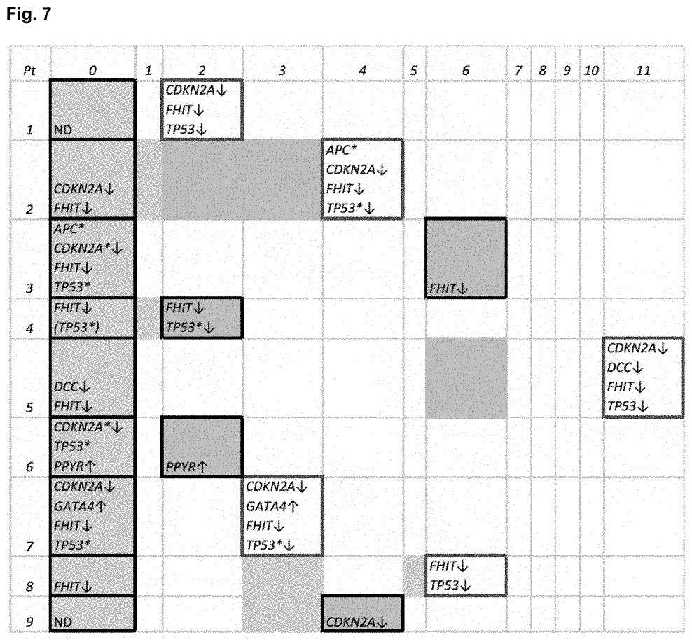

Overall, genomic alterations tested by both NGS and SNP arrays did not increase from pre-progression-BIM (88%) to concurrent-BIM (86%), including in five patients who had matched samples of pre-progression-BIM, concurrent-BIM and dysplasia/EAC lesions. FIG. 7 shows recurrent genomic alterations in nine patients with both pre-progression-BIM and subsequent dysplasia/EAC tested by NGS and SNP arrays. Six of the nine patients had FHIT deletions present in both pre-progression-BIM and subsequent dysplasia/EAC. CDKN2A mutations were present in both pre-progression-BIM and subsequent EAC in two of the nine cases. TP53 mutations were present in 3/9 pre-progression-BIM and TP53 SCNA and/or mutations were present in 5/9 dysplasia/EAC; however, in only 2 cases was the same mutation detected in both pre-progression-BIM and dysplasia/EAC (FIG. 7).

TABLE 7
Summary of genomic changes identified by NGS targeted panels (pathogenic mutations).

| | NDBE | | PP-BIM | | C-BIM | | DAC | |
|---|---|---|---|---|---|---|---|---|
| Pathogenic Mutations by NGS | (N = 27) | | (N = 16) | | (N = 19) | | (N = 13) | |
| CDKN2A | 1 | (4%) | 3 | (19%) | 4 | (21%) | 1 | (8%) |
| | | | [0.1179] | | [0.0789] | | [0.5972] | |
| TP53 | 0 | (0%) | 5 | (31%) | 4 | (21%) | 4 | (31%) |
| | | | [0.0037] | | [0.0171] | | [0.0039] | |
| APC | 0 | (0%) | 1 | (6%) | 2 | (11%) | 1 | (8%) |
| | | | [0.1939] | | [0.0918] | | [0.1495] | |
| NOTCH | 0 | (0%) | 0 | (0%) | 1 | (5%) | 0 | (0%) |
| | | | | | [0.2332] | | | |
| Any pathogenic mutation | 1 | (4%) | 6 | (38%) | 7 | (37%) | 5 | (38%) |
| | | | [0.0079] | | [0.0080] | | [0.0078] | |
| | NDBE | | B-BIMP | | C-BIMP | | DAC | |
| COMBINED NGS and SNP Arrays | (N = 21) | | (N = 14) | | (N = 19) | | (N = 13) | |
| Any CDKN2A or FHIT SCNA, Other | 5 | (24%) | 12 | (86%) | 17 | (89%) | 12 | (92%) |

| | | | |
|---|---|---|---|
| Deletion, or NGS Pathogenic Mutation | [0.0100] | [0.0084] | [0.0092] |

Note:
A subset of those samples had both NGS and Oncoscan SNP arrays performed, and the number of samples with any SCNA in CDKN2A or FHIT, or any somatic deletion or a pathogenic mutation are shown in the last row. See Table 3 for explanation of abbreviations and numbers in parenthesis.

TABLE 8
Results of NGS targeted panels.

| Group | Patient No | NGS Results |
|---|---|---|
| PP-BIM | 1-2, 4, 5, 9-11, | NMF (*) |
| | 13, 17, 18 | |
| PP-BIM | 3 | APC:NM_001127510:exon17:c.3949G > C:p.E1317Q (30.83%), cov = 959, COSM19099; |
| | | CDKN2A:NM_000077:exon2:c.304G > A:p.A102T (7.88%), cov = 416, COSM12740; |
| | | TP53:NM_001126114:exon6:c.646G > A:p.V216M (5.33%), cov = 1728, COSM3388195; |
| PP-BIM | 6 | CDKN2A:NM_000077:exon2:c.191C > T:p.L65P (11.1%), cov = 45, COSM13773; |
| | | TP53:NM_001126114:exon5:c.473G > A:p.R158H (6.16%), cov = 3196, COSM220780 |
| PP-BIM | 7 | TP53:NM_001126114:exon7:c.733G > A:p.G245S (12.44%), cov = 394, COSM3356965; |
| PP-BIM | 8 | TP53:NM_001126114:exon8:c.902C > A:p.P301Q (6.68%), cov = 1438, COSM43873; |
| PP-BIM | 12 | TP53:NM_001126114:exon8:c.796G > A.p.G266R (7.50%), cov = 2468, COSM10794 |
| PP-BIM | 14 | CDKN2A:NM_000077:exon2:c.174_181del:p.R58fs (34.09%), cov = 3785, noCosmic; |
| | | CDKN2A:NM_000077:exon2:c.172C > T:p.R58X (15.90%), cov = 1826, COSM99730; |
| NDBE | 19-35, 37-45 | NMF |
| NDBE | 38 | CDKN2A:NM_001195132:exon2:c.238C > T:p.R80X, cov = 1142, COSM1314729 |
| C-BIM | 6-8, 17, 49-53, | NMF # |
| | 56, 58, 59 | |
| C-BIM | 2 | APC:NM_001127510:exon17:c.3949G > C:p.E1317Q (21.70%), COSM19099 # |
| C-BIM | 4 | TP53:NM_001126114:exon6:c.637C > T:p.R213X (7.3%), cov = 3350, COSM99618 # |
| C-BIM | 46 | CDKN2A:NM_000077:exon2:c.163G > A:p.G55S (8.63%), cov = 139, noCosmic; |
| C-BIM | 48 | TP53:NM_001126114:exon7:c.743G > A:p.R248Q (9.52%), cov = 1964, COSM99602; |
| | | TP53.NM_001126114:exon6:c.578A > G:p.H193R (5.69%), cov = 2005, COSM308309 |
| C-BIM | 54 | NOTCH1:NM_017617:exon26:c.4715G > A.p.G1572D (12.63%), cov= 467, COSM5030961; |
| | | TP53:NM_001126114:exon8:c.878G > A:p.G293E (6.44%), cov = 621, noCosmic |
| C-BIM | 57 | TP53:NM_001126114:exon5:c.473G > T:p.R158L (11.08%), cov = 11275, COSM99677 |
| C-BIM | 60 | TP53:NM_001126114.2:exon6:c.577C > T:p.H193Y (35%) cov = 4851, COSM10672; |
| | | CDKN2A:NM_000077.3.exon2:c.250G > A:p.D64N (53%), cov = 1092, COSM13486; |
| | | APC:NM_001127510:exon16:c.3906_3907insA:p.L1302fs (22.38%), cov = 1975, noCosmic |
| DAC | 1, 3, 5, 6, 8, 9, | NMF |
| | 17, 61 | |
| DAC | 2 | APC:NM_001127510:exon17:c.3949G > C:p.E1317Q (47.03%), cov = 9167, COSM19099; |
| | | TP53:NM_001126114:exon8:c.916C > T:p.R306X (20.11%), cov = 2675, COSM10663 # |
| DAC | 4 | TP53:NM_001126114:exon6:c.637C > T:p.R213X (18%), cov = 3350, COSM99618 # |
| DAC | 7 | TP53:NM_001126114:exon7:c.733G > A.p.G245S (48.96%), cov = 2365, COSM3356965 |
| DAC | 62 | CDKN2A:NM_000077:exon2:c.172C > T:p.R58X (58%), cov = 131, COSM12473 |
| DAC | 63 | TP53:NM_001126114:exon8:c.839G > A:p.R280K (38%), cov = 3653, COSM10728 |

Note:
NMF: No mutations found. See Table 1 for other abbreviations. Description of mutations includes the following, separated by a colon: Gene symbol: Transcript GenBank number: exon number: cDNA base pair position in transcript followed by nucleotide change (reference > variant): aminoacid position in translated transcript following HGVS nomenclature (reference aminoacid - position - variant aminoacid): cov = coverage (total number of reads at variant position): COSMIC ID number (noCosmic: not present in COSMIC database).
* Patient 4 PP-BIM: TP53 R213X was present at 3.3%, but seen at much higher frequency in subsequent samples (C-BIM, DAC).
# NGS panel results in patients 19-22 and 42-45 (NDBE), and 2 and 4 (C-BIM and DAC) were previously reported (Del Portillo et al 2015).

### Example 5

### Genomic Alterations in FHIT Exon 5 and CDKN2A/B Represent Promising Biomarkers of Progression in Barrett's Esophagus Patients

We tested biopsies from two longitudinal cohorts: 1. Progressors, who had BIM samples described as pre-progression-BIM obtained before the diagnosis of dysplasia/EAC (median 43 months), and 2. Non-progressors, who had a diagnosis of BIM and did not progress to dysplasia/EAC (NDBE patients) during a median 51 months of follow-up from the tested biopsy.

SCNAs were more frequent than mutations in non-dysplastic BIM of progressors (88% vs. 38% in pre-progression-BIM and 81% vs. 37% in concurrent-BIM). The most frequent SCNAs in non-dysplastic BIM involved FHIT exon 5 and CDKN2A/B, and were significantly more prevalent in pre-progression-BIM (88%) than in NDBE (24%). Mutations were more frequent in pre-progression-BIM (38%) than NDBE (3%) but in pre-progression-BIM, NGS did not increase sensitivity for detecting genomic alterations compared to SNP-arrays alone. Importantly, detection of SCNAs in FHIT and CDKN2A/B by SNP-arrays had a sensitivity of 88% (95% C.I=71-100%) and specificity of 76% (95% C.I.=58-94%) for separating pre-progression-BIM of progressors from BIM of non-progressors, suggesting that the combined testing of FHIT and CDKN2A/B copy numbers may be useful as biomarkers in the surveillance stratification of BE patients. The specificity calculation assumes that none of the NDBE would progress to dysplasia/EAC.

Our data are consistent with previous studies showing frequent CDKN2A/p16 inactivation in EAC, pre-neoplastic BIM and dysplastic precursor lesions (Del Portillo et al 2015; Stachler et al 2015; Wang et al 2009). Similarly, altered FHIT transcripts have been described in the premalignant stages of EAC development (Michael et al 1997). About two thirds of patients in our study had FHIT deletion in the pre-progression-BIM that was present in subsequent dysplasia/EAC lesion. Our characterization of SCNAs and mutations in dysplastic lesions and EAC identified similar alterations as previously reported (Asan et al 2017), including frequent TP53 mutations (46%) and TP53 SCNAs (38%), in dysplasia/EAC lesions. However, no TP53 SCNAs were found in pre-progression-BIM and NDBE, and were found in only 14% of concurrent BIM, suggesting this alteration is a late event associated with progression to dysplasia/EAC.

Further, the number of genomic alterations did not increase from pre-progression to concurrent BIM, suggesting that “molecular dysplasia” in BIM at risk of progression can persist for several years, as assessed by genomic alterations in FHIT, CDKN2A and TP53 mutation, whereas TP53 LOH indicates progression to dysplasia/EAC. This is consistent with other recent studies (Weaver et al 2014; Stachler et al 2015; Li et al 2014). Performing both NGS and SNP-arrays detected genomic alterations in 86% of concurrent-BIM, suggesting that this combination of assays may be sensitive to detect concurrent dysplasia/EAC that may be missed in routine endoscopic surveillance.

In summary, our longitudinal study of Barrett's esophagus progression shows that genomic alterations primarily in FHIT exon 5 and CDKN2A/B are frequently detected in routine FFPE biopsy samples of non-dysplastic Barrett's epithelium of patients who harbor or develop future dysplasia or EAC, suggesting they represent biomarkers of progression that may be incorporated in routine workup for cancer surveillance in Barrett's esophagus patients.

### Example 5

Barrett's Esophagus Progression to Cancer (BPC) Gene Panel

The approach of a Barrett's Esophagus Progression to Cancer (BPC) Gene Panel is to sequence DNA extracted from tissue samples of Barrett's Esophagus (BE) and performing Next Generations Sequencing (NGS) using a custom designed panel of primers to amplify specific regions of the chromosome. The specific steps include:
(1) DNA extraction and purification from any tissue samples, including Formalin-Fixed Paraffin-Embedded (FFPE) or frozen tissue sections or blocks, using any method that yields at least 20 ng of adequate quality DNA.
(2) Library preparation using a custom designed set of primers.
   a. The target regions encompass the following:
      i. The complete coding sequence and selected intronic single-nucleotide polymorphisms (SNP) of the following genes, preferably from human:
         1. APC
         2. CDKN2A
         3. CDKN2B
         4. FHIT
         5. TP53
         Representative sequences of each of the above-identified genes may be found at, e.g., GenBank, under accession numbers: M74088.1 (APC), JQ694045.1 (CDKN2A), CR536529.1 (CDKN2B), BC032336.1 (FHIT), and KX710182.1 (TP53).
      ii. SNPs in 2-3 well-spaced regions of each arm of each of chromosomes 1 to 22.
   b. In the first iteration of primer design, 2001 primers were designed to sequence approximately a total 435 kb of the regions described above.
   c. The method for library preparation uses the Qiagen QIAseq Targeted Panel approach and kits. Briefly, the following steps are followed:
      i. DNA is randomly fragmented, end-repaired and A-tailed using an enzyme mix.
      ii. Oligonucleotides containing a common region and a unique universal molecular identifier (UMI) are attached to the 5′ end of each DNA molecule. The UMI comprises a 12-base fully random sequence and allows identification of each original molecule of DNA even after amplification by PCR.
      iii. Target enrichment is achieved by PCR using a universal forward primer and the target-specific primers described above. Using a single target specific primer rather than forward and reverse target specific primers provides less amplification errors due to primer interference during PCR.
      iv. Further amplification with a universal primer and a sample specific primer ensures a sequencing-ready library with enough DNA for sequencing and sample identification.
(3) Sequencing in an appropriate NGS sequencer; the IIlumina MiSeq was used.

### Bioinformatics Pipeline:

The Illumina pipeline produces FASTQ files which are then processed by the Qiagen SM-COUNTER algorithm to align them to the reference genome. Because each molecule is amplified with a different UMI, PCR duplicates are easily identified using algorithms such as SM-COUNTER and custom R/Bioconductor pipelines.

The goal of the analytical pipeline is to provide the following information:
(1) Presence of pathogenic mutations (including single nucleotide variants—SNV and small deletions or insertions) in the genes in the panel; this is accomplished by standard variant analysis and annotation, e.g. using the Qiagen SM-COUNTER pipeline. This will mark all the duplicate UMI reads, allowing for accurate variant allele frequency (VAF) determinations.
(2) VAF of all the SNPs detected. This will be plotted as the B-allele frequency=B/(B+A) to determine loss of heterozygosity (LOH) possibly associated with BE progression risk.
(3) Copy number variants (CNV) are assessed by counting UMI's associated with each targeted-specific primer corresponding to the initial number of DNA molecules of each target region present in the sample. Since the DNA is randomly fragmented, each region provides reads of different sizes. The analytical pipeline counts all target reads with a 5′-end mapping to the base following the primer sequence. Counts are then normalized by a number of statistical approaches, including comparison with non-affected areas of the genome and a set of region-specific count ranges established by analyzing several normal controls.

The presence of any of SNV, indels, LOH, and CNV in a region of interest is considered for estimation of the risk of progression of BE to dysplasia and esophageal adenocarcinoma.

### DOCUMENTS CITED

1. Pohl H, Sirovich B, Welch H G. Esophageal adenocarcinoma incidence: are we reaching the peak? Cancer Epidemiol Biomarkers Prey 2010; 19(6):1468-70.
2. Rustgi A K, EI-Serag H B. Esophageal carcinoma. N Engl J Med 2014; 371(26):2499-509.
3. Snider E J, Freedberg D E, Abrams J A. Potential Role of the Microbiome in Barrett's Esophagus and Esophageal Adenocarcinoma. Dig Dis Sci 2016; 61(8):2217-25.
4. Pohl J, Pech O, May A, et al. Incidence of macroscopically occult neoplasias in Barrett's esophagus: are random biopsies dispensable in the era of advanced endoscopic imaging? Am J Gastroenterol 2010; 105(11):2350-6.
5. Siegel R L, Miller K D, Jemal A. Cancer statistics, 2018. CA Cancer J Clin 2018; 68(1):7-30.
6. Seer.cancer.gov. Cancer Stat Facts: Esophageal Cancer. https://seer.cancer.gov/statfactsihtml/esoph.html.
7. Ruol A, Parenti A, Zaninotto G, et al. Intestinal metaplasia is the probable common precursor of adenocarcinoma in barrett esophagus and adenocarcinoma of the gastric cardia. Cancer 2000; 88(11):2520-8.
8. Shaheen N J, Falk G W, Iyer P G, et al. ACG Clinical Guideline: Diagnosis and Management of Barrett's Esophagus. Am J Gastroenterol 2016; 111(1):30-50; quiz 51.
9. American Gastroenterological A, Spechler S J, Sharma P, et al. American Gastroenterological Association medical position statement on the management of Barrett's esophagus. Gastroenterology 2011; 140(3):1084-91.
10. Ronkainen J, Aro P, Storskrubb T, et al. Prevalence of Barrett's esophagus in the general population: an endoscopic study. Gastroenterology 2005; 129(6):1825-31.
11. Sikkema M, de Jonge P J, Steyerberg E W, et al. Risk of esophageal adenocarcinoma and mortality in patients with Barrett's esophagus: a systematic review and meta-analysis. Clin Gastroenterol Hepatol 2010; 8(3):235-44; quiz e32.
12. Hvid-Jensen F, Pedersen L, Drewes A M, et al. Incidence of adenocarcinoma among patients with Barrett's esophagus. N Engl J Med 2011; 365(15):1375-83.
13. Buas M F, He Q, Johnson L G, et al. Germline variation in inflammation-related pathways and risk of Barrett's oesophagus and oesophageal adenocarcinoma. Gut 2016; 10.1136/gutjnI-2016-311622.
14. Desai T K, Krishnan K, Samala N, et al. The incidence of oesophageal adenocarcinoma in non-dysplastic Barrett's oesophagus: a meta-analysis. Gut 2012; 61(7):970-6.
15. Wani S, Falk G W, Post J, et al. Risk factors for progression of low-grade dysplasia in patients with Barrett's esophagus. Gastroenterology 2011; 141(4):1179-86, 1186 e1.
16. Wani S, Puli S R, Shaheen N J, et al. Esophageal adenocarcinoma in Barrett's esophagus after endoscopic ablative therapy: a meta-analysis and systematic review. Am J Gastroenterol 2009; 104(2):502-13.
17. Caygill C P, Dvorak K, Triadafilopoulos G, et al. Barrett's esophagus: surveillance and reversal. Ann N Y Acad Sci 2011; 1232:196-209.
18. Cancer Genome Atlas Research N, Analysis Working Group: Asan U, Agency BCC, et al. Integrated genomic characterization of oesophageal carcinoma. Nature 2017; 541(7636):169-175.
19. Dulak A M, Stojanov P, Peng S, et al. Exome and whole-genome sequencing of esophageal adenocarcinoma identifies recurrent driver events and mutational complexity. Nat Genet 2013; 45(5):478-86.
20. Secrier M, Li X, de Silva N, et al. Mutational signatures in esophageal adenocarcinoma define etiologically distinct subgroups with therapeutic relevance. Nat Genet 2016; 48(10): 1131-41.
21. Del Portillo A, Lagana S M, Yao Y, et al. Evaluation of Mutational Testing of Preneoplastic Barrett's Mucosa by Next-Generation Sequencing of Formalin-Fixed, Paraffin-Embedded Endoscopic Samples for Detection of Concurrent Dysplasia and Adenocarcinoma in Barrett's Esophagus. J Mol Diagn 2015; 17(4):412-9.
22. Agrawal N, Jiao Y, Bettegowda C, et al. Comparative genomic analysis of esophageal adenocarcinoma and squamous cell carcinoma. Cancer Discov 2012; 2(10):899-905.
23. Weaver J M, Ross-Innes C S, Shannon N, et al. Ordering of mutations in preinvasive disease stages of esophageal carcinogenesis. Nat Genet 2014; 10.1038/ng.3013.
24. Ross-Innes C S, Becq J, Warren A, et al. Whole-genome sequencing provides new insights into the clonal architecture of Barrett's esophagus and esophageal adenocarcinoma. Nat Genet 2015; 47(9):1038-46.
25. Stachler M D, Taylor-Weiner A, Peng S, et al. Paired exome analysis of Barrett's esophagus and adenocarcinoma. Nat Genet 2015; 47(9):1047-55.
26. Li X, Galipeau P C, Paulson T G, et al. Temporal and spatial evolution of somatic chromosomal alterations: a case-cohort study of Barrett's esophagus. Cancer Prey Res (Phila) 2014; 7(1):114-27.
27. Foster J M, Oumie A, Togneri F S, et al. Cross-laboratory validation of the OncoScan(R) FFPE Assay, a multiplex tool for whole genome tumour profiling. BMC Med Genomics 2015; 8:5.
28. Gliem T J, Aypar U. Development of a Chromosomal Microarray Test for the Detection of Abnormalities in Formalin-Fixed, Paraffin-Embedded Products of Conception Specimens. J Mol Diagn 2017; 19(6):843-847.
29. R Core Team. R: A Language and Environment for Statistical Computing}. In. Vienna, Austria: R Foundation for Statistical Computing; 2015.
30. Makishima H, Maciejewski J P. Pathogenesis and consequences of uniparental disomy in cancer. Clin Cancer Res 2011; 17(12):3913-23.
31. Wang J S, Guo M, Montgomery E A, et al. DNA promoter hypermethylation of p16 and APC predicts neoplastic progression in Barrett's esophagus. Am J Gastroenterol 2009; 104(9):2153-60.
32. Michael D, Beer D G, Wilke C W, et al. Frequent deletions of FHIT and FRA3B in

Barrett's metaplasia and esophageal adenocarcinomas. Oncogene 1997; 15(14):1653-9.
33. Ilumina Inc. Somatic Variant Caller. http://res.illurmina.com/documents/products/technotes/technote_somatic_variant caller.pdf.
34. Thorvaldsdottir H, Robinson J T, Mesirov J P. Integrative Genomics Viewer (IGV): high-performance genomics data visualization and exploration. Brief Bioinform 2013; 14(2):178-92.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

1. A method of predicting the risk of a subject having Barrett's Esophagus to develop a more severe condition, comprising:
a) obtaining a sample from the subject;
b) extracting DNA from the sample;
c) analyzing the DNA to detect a genomic alteration;
d) if the genomic alteration is detected, the subject is at high risk of developing a more severe condition; and
e) if the subject is determined to be at high risk, increasing the frequency of the subject's clinical screening.

2. The method of claim 1, wherein the analysis of DNA in step c) is performed with a Barrett's Esophagus Progression to Cancer (BPC) gene panel comprising the following genes: APC, CDKN2A, CDKN2B, FHIT and TP53.

3. The method of claim 1, wherein the more severe condition is selected from low-grade dysplasia (LGD), high-grade dysplasia (HGD) and esophageal adenocarcinoma (EAC).

4. The method of claim 1, wherein the sample is obtained from Barrett's tissue, cytology preparations, circulating cells, or blood.

5. The method of claim 1, wherein the sample is fresh or formalin fixed paraffin embedded (FFPE).

6. The method of claim 1, wherein the genomic alteration is selected from the group consisting of single nucleotide variants, complex insertions and deletions, genomic losses and gains, genome copy number changes, copy-neutral loss of heterozygosity, and combinations thereof.

7. The method of claim 1, wherein the genomic alteration is a somatic copy number alteration (SCNA).

8. The method of claim 7, wherein the SCNA is selected from deletion of FHIT including exon 5, hemizygous deletion of CDKN2A/2B with partial loss of heterozygosity (pLOH), other somatic deletions, and combinations thereof.

9. The method of claim 8, wherein the other somatic deletions is selected from the group consisting of hemizygous deletion of PRIM2 with pLOH, hemizygous deletion of chromosome 9p21.3-21.1 with pLOH, deletion of APC without pLOH, deletion of OVOS without pLOH, deletion of STYX without pLOH, deletion of DCC without pLOH, deletion of PLCB1 without pLOH, multiple deletions of MTOR, chromosome 5q22.2, chromosome 9p13.3, OVOS, SPRED1, SMAD7, PLCB1 and PLA2G3, and combinations thereof.

10. The method of claim 1, wherein the genomic alteration is a pathogenic mutation.

11. The method of claim 10, wherein the pathogenic mutation is a TP53 mutation.

12. The method of claim 7, wherein the SCNA is detected by genome-wide single nucleotide polymorphism (SNP) arrays.

13. The method of claim 10, wherein the pathogenic mutation is detected by targeted next generation sequencing (NGS) with a cancer panel.

14. The method of claim 13, the cancer panel is a TruSeq cancer panel comprising the following cancer genes: ABL1, AKT1, ALK, APC, ATM, BRAF, CDH1, CDKN2A, CSF1R, CTNNB1, EGFR, ERBB2, ERBB4, FBXW7, FGFR1, FGFR2, FGFR3, FLT3, GNA11, GNAS, GNAQ, HNF1A, HRAS, JAK2, JAK3, IDH1, KDR/VEGFR2, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, and VHL.

15. The method of claim 13, the cancer panel is an AmpliSeq cancer panel comprising the following cancer genes: ABL1, AKT1, ALK, APC, ATM, BRAF, CDH1, CDKN2A, CSF1R, CTNNB1, EGFR, ERBB2, ERBB4, EZH2, FBXW7, FGFR1, FGFR2, FGFR3, FLT3, GNA11, GNAS, GNAQ, HNF1A, HRAS, JAK2, JAK3, IDH1, IDH2, KDR/VEGFR2, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, and VHL.

16. A method of predicting the risk of a subject having Barrett's Esophagus to develop dysplasia and/or esophageal adenocarcinoma (EAC), comprising:
a) obtaining a formalin fixed paraffin embedded (FFPE) biopsy sample from the subject;
b) extracting DNA from the sample;
c) analyzing the DNA to detect a genomic alteration, wherein the genomic alteration is somatic copy number alterations (SCNAs) in FHIT exon 5 and CDKN2A/2B;
d) if the genomic alteration is detected, the subject is at high risk of developing dysplasia and/or EAC; and
e) if the subject is determined to be at high risk, increasing the frequency of the subject's clinical screening.

17. The method of claim 16, wherein the analysis of DNA in step c) is performed with a Barrett's Esophagus Progression to Cancer (BPC) gene panel comprising the following genes: APC, CDKN2A, CDKN2B, FHIT and TP53.

18. The method of 16, wherein the genomic alteration is a combination of SCNAs in FHIT exon 5 and CDKN2A/2B plus a TP53 mutation.

19. A method of supporting the diagnosis of dysplasia or esophageal adenocarcinoma (EAC) in a subject having Barrett's Esophagus, comprising:
a) obtaining a sample from the subject;
b) extracting DNA from the sample;
c) analyzing the DNA by both genome-wide single nucleotide polymorphism (SNP) arrays and next generation sequencing (NGS)to detect a genomic alteration;
d) if the genomic alteration is detected, the subject is likely to have dysplasia or EAC; and
e) treating the diagnosed subject for dysplasia or EAC.

20. The method of claim 19, wherein the analysis of DNA in step c) is performed with a Barrett's Esophagus Progression to Cancer (BPC) gene panel comprising the following genes: APC, CDKN2A, CDKN2B, FHIT and TP53.

21-30. (canceled)
